(19) United States
(12) Patent Application Publication
(10) Pub. No.: US 2026/0285794 A1
(43) Pub. Date: Sep. 24, 2026

(54) PROCESSES FOR THE PREPARATION OF CITRAL AND INTERRELATED PRODUCTS

(71) Applicant: BASF SE, Ludwigshafen am Rhein (DE)

(72) Inventors: Martin KAMASZ, Ludwigshafen am Rhein (DE); Andreas KELLER, Ludwigshafen am Rhein (DE); Gunnar HEYDRICH, Ludwigshafen am Rhein (DE); Bernhard BRUNNER, Ludwigshafen am Rhein (DE); Rupert WAGNER, Ludwigshafen am Rhein (DE); Nicolas DUYCKAERTS, Ludwigshafen am Rhein (DE); Christian WALSDORFF, Ludwigshafen am Rhein (DE); Volker Gerhard DINGES, Shanghai (CN); Lee Russell COLLINS, Ludwigshafen am Rhein (DE)

(21) Appl. No.: 19/489,329

(22) PCT Filed: May 31, 2024
(86) PCT No.: PCT/EP2024/065012

§ 371 Date: Dec. 2, 2025

(30) Foreign Application Priority Data
Jun. 2, 2023 (EP) 23177047.0
Jan. 17, 2024 (EP) 24152476.8

(51) Int. Cl. C07C 45/67; C07C 41/56; C07C 45/38

(52) U.S. Cl. CPC C07C 45/673; C07C 41/56; C07C 45/38

(57) ABSTRACT

A shell-and-tube heat exchange reactor for carrying out a catalytic gas-phase partial oxidation reaction comprises a shell-side heat exchange passage for circulating a heat transfer medium and a reaction passage comprising a plurality of reaction tubes; an inlet for introducing the reactant stream to the reaction passage; and an outlet from the reaction passage for recovering an effluent stream from the reaction tubes. The reaction tubes comprise a reactant pre-heating zone adjacent to the inlet, and a reaction zone downstream of the reactant pre-heating zone, the reaction zone having a catalytically active wire matrix insert having at least on a part of its surface a catalytically active precious metal. The reactor requires less frequent maintenance in the form of regeneration and/or replacement of the catalyst. The catalyst can be easily placed into the reaction tubes, and be removed therefrom. Only the portion of the entire reactant stream that travels near the hot reaction tube wall is heated up. Consequently, the portion of the reactant stream flowing in the center of the reaction tube is not heated to the reaction temperature and blind reactions of the unstable starting materials are thus reduced or even avoided.

### FIELD OF THE INVENTION

The present invention relates to processes for preparing 3-methyl-2-butenal (prenal), for preparing 3-methyl-2-buten-1-ol (prenol), for preparing diprenyl acetate of prenal as well as for preparing 3,7-dimethyl-octa-2,6-dienal (citral), wherein the aldehyde contents in one or more reactant streams are maintained to be below thresholds. Furthermore, the present invention relates to obtainable product streams of prenal, prenol, diprenyl acetate of prenal as well as citral. In particular, the present invention refers to such processes using isoprenol as educt. Thus, the present invention relates to an improved process for preparing prenol from isoprenol, for preparing prenal from isoprenol, for preparing p diprenyl acetate of prenal from isoprenol, as well as to an improved process for the preparation of citral from isoprenol.

According to the invention, a process for the preparation of 3,7-dimethyl-octa-2,6-dienal (citral) comprises reacting at least one formaldehyde source and isobutylene to obtain isoprenol (step a). Isoprenol obtained in step a) is isomerized to obtain prenol by bringing a reactant stream comprising isoprenol into contact with at least one heterogeneous isomerization catalyst (step b). Further, prenal is provided by at least one of steps c-i) and c-ii): c-i) subjecting isoprenol obtained in step a) to oxidative dehydrogenation so as to obtain prenal and/or isoprenal; c-ii) oxidizing prenol obtained in step b) so as to obtain prenal. Moreover, prenol obtained in step b) is condensed with prenal obtained in step c) to obtain diprenyl acetal of prenal (step d). Further, diprenyl acetal of prenal obtained in step d) is subjected to cleaving conditions to obtain citral via prenyl (3-methyl-butadienyl) ether and 2,4,4-trimethyl-3-formyl-1,5-hexadiene (step e). Step c-i) is characterized by maintaining in the reactant stream a weight ratio of aldehydes to isoprenol of less than 0.04.

### BACKGROUND OF THE INVENTION

Prenal, prenol, diprenyl acetate of prenal, and citral are chemical compounds that may be used as products or as educts for numerous further reactions. The aforementioned chemical compounds may be obtained from reactions using isoprenol as educt and/or intermediate product.

Prenol is sought after as an intermediate for the synthesis of scents, vitamins and carotenoids. Its preparation by isomerization of isoprenol over suitable catalysts is known to those skilled in the art and has been widely described in the literature.

Isoprenol can be isomerized to 3-methyl-2-buten-1-ol (prenol) in the presence of hydrogen and a catalyst, the catalyst used being in particular a fixed-bed catalyst containing palladium and selenium or tellurium or a mixture of selenium and tellurium on a silica support. From J. Am. Chem. Soc., 85 (1963), pages 1549 to 1550, the isomerization of an unsaturated alcohol with a carbonyl compound of a metal of group VIII. Group of the Periodic Table of the Elements catalyst is well known. This process yields numerous by-products and derivatives, for example the corresponding aldehydes.

DE-C-19 01 709 describes a process for the preparation of butene-2-ol-4 compounds in which butene-1-ol-4 compounds are reacted in the presence of palladium or palladium compounds and hydrogen. However, when pure palladium is used in the presence of hydrogen, the double bond of the compounds is hydrogenated to a considerable extent and a saturated product is formed.

In addition, low-boiling compounds such as hydrocarbons and aldehydes are formed as by-products, for example by hydrogenolysis and isomerization. Hydrogenation of the double bond is undesirable, since for some butenols there are only slight differences in boiling point between the unreacted starting product and the hydrogenation product. For example, the boiling point of 3-methyl-3-buten-1-ol at 1020 mbar is 131.5° C., while the boiling point of the corresponding hydrogenation product, 3-methyl-1-butanol, is 130.9° C. This makes it difficult to separate the hydrogenation product from the starting product by distillation.

A process for the isomerization of 3-buten-1-ol compounds to the corresponding 2-buten-1-ol compounds is known from DE-A 27 51 766, the isomerization being carried out in the presence of palladium and selenium or tellurium as catalyst and hydrogen. Palladium and selenium on activated carbon is used as catalyst. Other supports that can be used include barium sulfate, silica gel, alumina and zeolites. The catalysts can also be used without supports. Relatively high proportions of light boilers, such as isoprene and methylbutenes, are formed. The known catalytic isomerizations are carried out discontinuously, for example in a stirred tank in suspension mode. Since the double-bond isomerization of substituted butenols is an equilibrium reaction, however, no complete conversion of substances is obtained; instead, a portion of the starting material always remains, which must be separated from the by-products formed for further use. In order to carry out the isomerization more economically, it should be possible to carry out the reaction continuously and it should lead to a minimum proportion of hydrogenation products or low boilers.

EP-A 841 090 describes a process for the continuous production of 2-buten-1-ol compounds by isomerization of 3-buten-1-ol compounds, the amount of hydrogenation products and light boilers being very low. The catalyst used is a fixed-bed catalyst containing palladium and selenium or tellurium or a mixture of selenium and tellurium on a silica support and having a BET surface area of 80 to 380 m²/g and a pore volume of 0.6 to 0.95 cm³/g in the pore diameter range from 3 nm to 300 μm, 80 to 95% of the pore volume being in the pore diameter range from 10 to 100 nm.

There is an ongoing need for processes which maintain high conversion and selectivity over time and avoid catalyst deactivation.

Prenol plays an important role in the production of citral.

Citral is a valuable intermediate for the production of various odorants and fragrances, such as geraniol. In addition, citral has also gained importance as a starting material for the production of vitamins, especially vitamin A.

WO 2008/037693 discloses a method for producing citral. Said method involves the following steps:
a) 3-methyl-3-butene-1-ol (isoprenol) is produced from isobutylene and formaldehyde;
b) 3-methyl-2-butenal (prenal) and 3-methyl-3-butenal (isoprenal) are produced from 3-methyl-3-butene-1-ol (isoprenol) by oxidative dehydrogenation by means of an oxygen-containing gas on a silver support catalyst;
c) additional 3-methyl-2-butenal (prenal) is produced from a mixture containing 3-methyl-3-butenal (isoprenal) by isomerization;
d) 3-methyl-2-butene-1-ol (prenol) is produced from 3-methyl-3-butene-1-ol (isoprenol) by isomerization;
e) the unsaturated acetal 3-methyl-2-butenal-diprenylacetal is produced from 3-methyl-2-butene-1-ol (prenol) and 3-methyl-2-butenal (prenal) using an acidic catalyst; and
f) 3,7-dimethyl-octa-2,6-diene-al (citral) is obtained from 3-methyl-2-butenal-diprenylacetal by cleavage and subsequently rearranging.

This complex, multi-stage process is prone to unwanted side reactions that reduce the attainable citral yield. Stated otherwise, the individual steps exhibit a less than 100% selectivity and the quantity of by-products formed may be higher than desired. Such by-products reduce the desired selectivity of the conversion and generally must be removed from the citral product prior to subsequent use. A substantial amount of energy is required to separate by-products from citral and significant losses of citral normally are encountered. Such losses may render the use of an otherwise advantageous reaction sequence commercially unattractive.

There is still an unmet desire for processes of preparing prenal, prenol, diprenyl acetate of prenal, and citral at improved selectivity and/or improved yields.

### SUMMARY OF THE INVENTION

Surprisingly, processes for the preparation of prenal, prenol, diprenyl acetate of prenal as well as citral may be improved by maintaining aldehyde contents below rather low thresholds in one or more reactant streams, in particular one or more reactant streams when conducting oxidative dehydrogenation of isoprenol to prenal and/or isoprenal and/or when conducting isomerization of isoprenol to prenol.

In a first aspect, it is an object of the invention to provide an improved process for the preparation of 3-methyl-2-butene-1-ol (prenol) from 3-methyl-3-butene-1-ol (isoprenol) by isomerization which maintains high conversion and selectivity over time and avoid catalyst deactivation.

Surprisingly, it has now been found that the presence of aldehydes, especially formaldehyde and/or prenal, in the reactant stream is detrimental to the activity and selectivity of the process and may accelerate catalyst deactivation and/or poisoning in the isomerization of isoprenol to prenol.

In the first aspect, the invention therefore relates to a process for the preparation of 3-methyl-2-butene-1-ol (prenol) by bringing a reactant stream comprising 3-methyl-3-butene-1-ol (isoprenol) into contact with at least one heterogeneous isomerization catalyst, preferably in the presence of hydrogen, to obtain a prenol containing product stream, characterized by maintaining in the reactant stream a concentration of aldehydes of less than 0.5 wt.-%, preferably less than 0.4 wt.-%, in particular less than 0.3 wt.-%, or less than 0.25 wt.-%, based on the total weight of the reactant stream, wherein the concentration of aldehydes in the reactant stream is not lower than 10 ppm, preferably not lower than 25 ppm, in particular not lower than 50 ppm, or not lower than 100 ppm, with respect to the total weight of the reactant stream.

In still more preferred embodiments, the concentration of aldehydes is maintained at less than 0.2 wt.-%, based on the total weight of the reactant stream, wherein the concentration of aldehydes in the reactant stream is not lower than 10 ppm, preferably not lower than 25 ppm, in particular not lower than 50 ppm, or not lower than 100 ppm, with respect to the total weight of the reactant stream.

In a second aspect, it is an object of the invention to advise an improved process for the preparation of citral.

In general terms, the invention provides a process for the preparation of 3,7-dimethyl-octa-2,6-dienal (citral) comprising the steps of:
a) reacting at least one formaldehyde source and isobutylene to obtain isoprenol;
b) isomerizing isoprenol to obtain prenol;
c) converting isoprenol or prenol to prenal, involving isomerization and an oxidative dehydrogenation in any order;
d) condensing prenol with prenal to obtain diprenyl acetal of prenal;
e) subjecting diprenyl acetal of prenal to cleaving conditions to obtain citral via prenyl (3-methyl-butadienyl) ether and 2,4,4-trimethyl-3-formyl-1,5-hexadiene.

In particular, the second aspect of the invention provides a process for the preparation of 3,7-dimethyl-octa-2,6-dienal (citral) comprising the steps of:
a) reacting at least one formaldehyde source and isobutylene to obtain isoprenol;
b) isomerizing isoprenol obtained in step a) to obtain prenol by bringing a reactant stream comprising isoprenol into contact with at least one heterogeneous isomerization catalyst, preferably in the presence of hydrogen;
c) providing prenal by at least one of c-i) and c-ii):
   c-i) subjecting isoprenol obtained in step a) to oxidative dehydrogenation so as to obtain prenal and/or isoprenal by bringing a reactant stream comprising isoprenol into contact with at least one heterogeneous oxidative dehydrogenation catalyst, in the presence of molecular oxygen, and optionally isomerizing at least part of the isoprenal to prenal;
   c-ii) oxidizing prenol obtained in step b) so as to obtain prenal by bringing a reactant stream comprising prenol into contact with at least one oxidant and at least one oxidation catalyst, preferably in the presence of a liquid phase;
d) condensing prenol obtained in step b) with prenal obtained in step c) to obtain diprenyl acetal of prenal; and
e) subjecting diprenyl acetal of prenal obtained in step d) to cleaving conditions to obtain citral via prenyl (3-methyl-butadienyl) ether and 2,4,4-trimethyl-3-formyl-1,5-hexadiene.

The process meets the following condition 1), and preferably the following condition 2), or the process meets at least one of the following conditions 1) and 2):
1) Step c-i) is characterized by maintaining in the reactant stream a weight ratio of aldehydes to isoprenol of less than 0.04.
2) Step b) is characterized by maintaining in the reactant stream a concentration of aldehydes of less than 0.5 wt.-%, preferably less than 0.4 wt.-%, in particular less than 0.3 wt.-%, or less than 0.25 wt.-%, based on the total weight of the reactant stream, and, optionally, the concentration of aldehydes in the reactant stream is not lower than 10 ppm, preferably not lower than 25 ppm, in particular not lower than 50 ppm, or not lower than 100 ppm, based on the total weight of the reactant stream.

In a further aspect, it is an object of the invention to provide an improved process for the preparation prenal.

In general terms, the invention provides a process for the preparation of prenal comprising a step of subjecting isoprenol to oxidative dehydrogenation so as to obtain prenal and/or isoprenal by bringing a reactant stream comprising isoprenol into contact with at least one heterogeneous oxidative dehydrogenation catalyst, in the presence of molecular oxygen, and optionally isomerizing at least part of the isoprenal to prenal, wherein the step is characterized by maintaining in the reactant stream a weight ratio of aldehydes to isoprenol of less than 0.04.

As surprisingly found experimentally, such process for the preparation of prenal is particularly beneficial. It may provide high selectivity of the reaction to prenal while maintaining high yields.

A still further aspect of the present invention relates to an improved process for the preparation of diprenyl acetal of prenal.

In general terms, the invention provides a process for the preparation of diprenyl acetal of prenal comprising the step of condensing prenal with prenol, wherein at least one of prenal and/or prenol is obtainable (or obtained) according to a process of the present invention.

As evident from experimental findings, such process for the preparation of diprenyl acetal of prenal is particularly beneficial. It may provide high selectivity of the reaction to diprenyl acetal of while maintaining high yields.

Preferably, when preparing prenal, prenol, diprenyl acetal of prenal or citral, the isoprenol may be obtained from reacting at least one formaldehyde source and isobutylene so as to obtain the isoprenol.

Further aspect of the present invention refers to product streams of prenal, prenol, diprenyl acetal of prenal, and citral obtainable (or obtained) from a process of the present invention. In a preferred embodiment, a product stream containing prenol is obtainable (or obtained) according to any one of claims 20 to 25. In a preferred embodiment, a product stream containing prenal is obtainable (or obtained) according to any one of claims 1, 2 and 13 to 16. In a preferred embodiment, a product stream containing diprenyl acetal of prenal is obtainable (or obtained) according to any one of claims 3 to 7, 10 to 17 and 26 to 30. In a preferred embodiment, a product stream containing citral is obtainable (or obtained) according to any one of claims 8 to 19, 31 and 32. As experimentally evidenced, the product streams may contain the respective products in particularly high selectivity. Thus, the obtainable (or obtained) products may bear special and technically beneficial characteristics.

### Definitions

As used herein and hereinafter, the term “concentration of aldehydes in the reactant stream” refers to the total concentration of aldehydes existing in the reactant stream. Aldehydes include those intrinsic to the isoprenol preparation process and those formed by oxidation and isomerization. Hence, the aldehydes usually include formaldehyde and/or prenal. In one embodiment, the aldehydes of which the concentration is determined are the sum of formaldehyde and prenal. Therefore, if formaldehyde and prenal are the only aldehydes existing in the reactant stream, the concentration of aldehydes in the reactant stream is the sum of the respective concentrations of formaldehyde and prenal.

As used herein and hereinafter, the term “ppm” refers to parts-per-million (ppm, 10-6).

As used herein, “formaldehyde source” refers to any source containing formaldehyde or capable of cleaving off formaldehyde. Formaldehyde sources include aqueous formaldehyde solutions and oligomers or polymers of formaldehyde, like paraformaldehyde.

Here and throughout the specification, the terms “wt.-%”, “wt. %”, “wt %”, “weight percent” and “% by weight” are used synonymously.

As used herein and hereinafter, the term “reactant stream” refers to a stream comprising a reactant or reactants consumed in the course of a chemical reaction. In this sense, the reactant stream may further comprise solvent(s), catalyst(s), additive(s) and/or any other substance involved in the chemical reaction.

As used herein and hereinafter, the term “unreacted isoprenol stream” refers to a stream which is derived from an isoprenol isomerization process and comprises unreacted isoprenol of the isoprenol isomerization process. In this sense, the unreacted isoprenol stream may further comprise solvent(s), catalyst(s), additive(s) and/or any other substance involved in the isoprenol isomerization process.

As used herein and hereinafter, the term “crude isoprenol stream” refers to a product stream of an isoprenol production process from which unreacted isobutylene has been removed. Removal of aldehydes, such as formaldehyde and/or prenal, is accomplished in a purification unit following the isoprenol synthesis. A preferred method of recovering aldehydes from a crude isoprenol stream to which an unreacted isoprenol stream is admixed, is described in more detail below.

### DETAILED DESCRIPTION OF THE INVENTION

The first aspect and the second aspect of the invention are discussed in more detail in the following.

### First Aspect of the Invention: Isomerization of Isoprenol

It has been found that deterioration of catalyst properties is related to the presence of aldehydes, especially formaldehyde and/or prenal in the reactant stream. Formaldehyde is generally considered to be the most critical of these aldehydes. Catalyst-fouling reactions of condensation and polymerization are believed to be the principal reactions involved in carbon or coke formation on the catalyst. It is thought that this carbon formation involves thermal condensation of aldehydes, for example formaldehyde and/or prenal, or of these aldehydes with the olefinic hydrocarbons isoprenol. In the presence of the catalyst the primary condensation products tend to undergo dehydrogenation and polymerization type reactions and to settle on the catalyst and undergo further dehydrogenation and decomposition until carbonaceous deposits are formed.

One of the poisoning mechanisms of the catalyst is supposed to involve a catalytic or non-catalytic dehydrogenation of aldehydes, especially formaldehyde and/or prenal to carbon monoxide, which is chemisorbed on the catalyst and blocks the active centers.

A further cause of catalyst deactivation, which may occur in combination with the previously mentioned cause of catalyst poisoning, is the formation of paraformaldehyde or trioxane which may deposit, in the form of solids, on the catalyst and shield the catalytically active surfaces from the isoprenol being processed. This leads to progressive deactivation of the catalyst.

According to the invention, the concentration of aldehydes in the reactant stream is, therefore, maintained at a certain level or less, i.e., less than 0.5 wt.-%, preferably less than 0.4 wt.-%, in particular less than 0.3 wt.-%, or less than 0.25 wt.-%, based on the total weight of the reactant stream, wherein the concentration of aldehydes in the reactant stream is not lower than 10 ppm, preferably not lower than 25 ppm, in particular not lower than 50 ppm, or not lower than 100 ppm, with respect to the total weight of the reactant stream.

In still more preferred embodiments, the concentration of aldehydes is maintained at less than 0.2 wt.-%, based on the total weight of the reactant stream, wherein the concentration of aldehydes in the reactant stream is not lower than 10 ppm, preferably not lower than 25 ppm, in particular not lower than 50 ppm, or not lower than 100 ppm, with respect to the total weight of the reactant stream.

Preferably, the aldehydes existing in the reactant stream comprise formaldehyde. Also preferably, the aldehydes existing in the reactant stream comprise prenal besides formaldehyde.

More preferably, the aldehydes existing in the reactant stream consist of prenal and formaldehyde. In certain instances, the aldehydes existing in the reactant stream consist of formaldehyde.

Preferably, the concentration of aldehydes in the reactant stream is less than 0.5 wt.-%, or 0.4 wt.-%, or 0.3 wt.-%, more preferably less than 0.25 wt.-%, or 0.2 wt.-%, even more preferably less than 0.15 wt.-%, yet even more preferably less than 0.1 wt.-%, equal to or less than 0.08 wt.-% or less than 0.05 wt.-%, based on the total weight of the reactant stream, but at least 10 ppm with respect to the total weight of the reactant stream. In another embodiment, the concentration of aldehydes is less than 0.025 wt.-%, more less than 0.02 wt.-%, based on the total weight of the reactant stream. In one embodiment, the concentration of aldehydes in the reactant stream is not lower than 10 ppm, preferably not lower than 25 ppm, in particular not lower than 50 ppm, or not lower than 100 ppm, with respect to the total weight of the reactant stream. The skilled person will appreciate that any of the upper limits of aldehyde concentration can be combined with any of the lower limits of aldehyde concentration, wherein in certain embodiments the aldehyde is either formaldehyde, prenal, or formaldehyde and prenal.

Also preferably, the concentration of formaldehyde in the reactant stream is less than 0.5 wt.-%, or 0.4 wt.-%, or 0.3 wt.-%, more preferably less than 0.25 wt.-%, or 0.2 wt.-%, even more preferably less than 0.15 wt.-%, yet even more preferably less than 0.1 wt.-%, equal to or less than 0.08 wt.-% or less than 0.05 wt.-%, based on the total weight of the reactant stream, but at least 10 ppm with respect to the total weight of the reactant stream. In another embodiment, the concentration of formaldehyde is less than 0.025 wt.-%, more less than 0.02 wt.-%, based on the total weight of the reactant stream, wherein the concentration of formaldehyde in the reactant stream is not lower than 10 ppm, preferably not lower than 25 ppm, in particular not lower than 50 ppm, or not lower than 100 ppm, with respect to the total weight of the reactant stream.

If the aldehydes existing in the reactant stream comprise or consist of formaldehyde, the concentration of formaldehyde in the reactant stream is preferably less than 0.5 wt.-%, or less than 0.4 wt.-%, or less than 0.3 wt.-%, more preferably less than 0.25 wt.-%, or 0.2 wt.-%, even more preferably less than 0.15 wt.-%, yet even more preferably less than 0.1 wt.-%, or less than 0.05 wt.-%, most preferably less than 0.025 wt.-%, or less than 0.02 wt.-%, based on the total weight of the reactant stream, wherein the concentration of aldehydes in the reactant stream is not lower than 10 ppm, preferably not lower than 25 ppm, in particular not lower than 50 ppm, or not lower than 100 ppm, with respect to the total weight of the reactant stream.

Preferably, the concentration of prenal in the reactant stream is less than 0.3 wt.-%, more preferably less than 0.2 wt.-%, even more preferably less than 0.15 wt.-%, in particular less than 0.1 wt.-%, based on the total weight of the reactant stream, but not lower than 10 ppm, preferably not lower than 25 ppm, in particular not lower than 50 ppm, or not lower than 100 ppm, with respect to the total weight of the reactant stream.

Therefore, in preferred embodiments, the aldehydes in the reactant stream consist of or comprises formaldehyde, and the concentration of formaldehyde is less than 0.5 wt.-%, or less than 0.4 wt.-%, or less than 0.3 wt.-%, more preferably less than 0.25 wt.-%, or 0.2 wt.-%, even more preferably less than 0.15 wt.-%, yet even more preferably less than 0.1 wt.-%, equal to or less than 0.08 wt.-%, or less than 0.05 wt.-%, based on the total weight of the reactant stream, but at least 10 ppm with respect to the total weight of the reactant stream. In another embodiment, the concentration of formaldehyde is less than 0.025 wt.-%, more preferably less than 0.02 wt.-%, based on the total weight of the reactant stream, but not less than 10 ppm, preferably not lower than 25 ppm, in particular not lower than 50 ppm, or not lower than 100 ppm, with respect to the total weight of the reactant stream. In one embodiment, the concentration of formaldehyde is equal to or less than 0.08 wt.-%, based on the total weight of the reactant stream, but optionally at least 10 ppm with respect to the total weight of the reactant stream.

In an embodiment, the aldehydes existing in the reactant stream consist of prenal and formaldehyde, and therefore the concentration of aldehydes in the reactant stream corresponds to the sum of the concentrations of prenal and formaldehyde, wherein the concentration of aldehydes in the reactant stream, i.e., the sum of the concentrations of prenal and formaldehyde is less than 0.5 wt.-%, preferably less than 0.4 wt.-%, in particular less than 0.3 wt.-% or less than 0.2 wt.-%, based on the total weight of the reactant stream, wherein the concentration of aldehydes in the reactant stream is not lower than 10 ppm, preferably not lower than 25 ppm, in particular not lower than 50 ppm, or not lower than 100 ppm, with respect to the total weight of the reactant stream.

In a group of the preferred embodiments, the weight ratio of aldehydes, preferably prenal and/or formaldehyde, to isoprenol in the reactant stream is adjusted at a certain level or less, i.e., less than 0.04, preferably less than 0.03, in particular less than 0.02, or less than 0.01. In still more preferred embodiments, the weight ratio of aldehydes, preferably formaldehyde and/or prenal, to isoprenol is adjusted at less than 0.002, or less than 0.001. In one embodiment, the ratio is lower than 0.0009.

The terms “maintaining in the reactant stream” and “adjusting in the reactant stream” or “maintained in the reactant stream” or “adjusted in the reactant stream” with respect to the aldehyde levels in the reactant stream are used interchangeably herein.

Reducing the weight ratio of aldehydes, preferably formaldehyde and/or prenal to isoprenol in the reactant stream beyond a certain point, however, reaches a point of rapidly diminishing return. Removal of aldehydes, especially of formaldehyde and/or prenal involves additional equipment and operating costs. An economic balance must be taken between the improvement due to reducing the ratio and the cost of maintaining such a ratio.

Hence, the weight ratio of aldehydes, preferably formaldehyde and/or prenal to isoprenol is preferably not lower than 0.0005 or, in some instances, not lower than 0.0007.

Since the double-bond isomerization of isomerization of isoprenol to prenol is an equilibrium reaction, a complete conversion of substances is not achieved in a single pass. Instead, a portion of isoprenol always remains, which unreacted isoprenol is suitably separated from the desired prenol. The unreacted isoprenol may be recycled to the isomerization reaction, or may be directed to other isoprenol-consuming reactions.

Generally, the reactant stream will comprise or consist of a fresh isoprenol stream. The term “fresh isoprenol stream” refers to a stream of isoprenol directly obtained from the purification unit following the isoprenol synthesis, i.e., from a purification unit wherein a crude isoprenol stream from the reaction of isobutene and formaldehyde is purified. The reactant stream may further comprise recycled, unreacted isoprenol, and/or isoprenol from other sources.

Preferably, the reactant stream comprises or consists of a fresh isoprenol stream. Also preferably, the reactant stream comprises or consists of a mixture of unreacted isoprenol stream and a fresh isoprenol stream.

In yet another embodiment, the reactant stream consists of a mixture of the unreacted isoprenol stream, and isoprenol from other sources. Other sources of isoprenol are processes other than the reaction of isobutene and formaldehyde, in which isoprenol is obtained as a by-product or target product, or isoprenol from commercial sources.

The presence of aldehydes, especially formaldehyde and/or prenal in the reactant stream reduces both catalyst activity and selectivity and causes increase in pressure drop and reactor clogging. Besides aldehydes, especially formaldehyde and/or prenal, other impurities which may be present in the reactant stream can cause a decrease in catalyst activity and selectivity. Preferably, the equipment or operations used for maintaining in the reactant stream a certain concentration of aldehydes, preferably formaldehyde and/or prenal, or a certain weight ratio of aldehydes, preferably formaldehyde or prenal to isoprenol is also effective to remove a major portion of these impurities. In preferred embodiments, the concentration in the reactant stream of at least one of the following impurities is kept below the limit indicated, in particular of all of the following impurities:

| | Concentration limit (wt.-%), based on |
|---|---|
| Impurity | the total weight of the stream |
| Isoamyl alcohol | 0.05 |
| Prenol | 0.20 |

Compliance with these limits is particularly important when the reactant stream accommodates isoprenol streams from other sources.

Reducing the concentration of aldehydes, preferably formaldehyde and/or prenal in the reactant stream will inherently reduce the weight ratio of aldehydes, preferably formaldehyde and/or prenal to isoprenol in the reactant stream. Therefore, the following applies for reducing the concentration of aldehydes, preferably formaldehyde and/or prenal in the reactant stream as well as reducing the weight ratio of aldehydes, preferably formaldehyde and/or prenal to isoprenol in the reactant stream.

The presence of formaldehyde in the reactant stream is due to two main sources. Formaldehyde may be contained in the isoprenol stream sent to the reactor, that is as an impurity originating from the isoprenol manufacture step. In industrial practice, isoprenol is synthesized from isobutene and formaldehyde. All the formaldehyde that cannot be separated in the purification step following the isoprenol synthesis ends up in the reactant stream.

In addition, formaldehyde is also generated in situ. Part of the isoprenol splits back to isobutene and formaldehyde.

Since most continuous industrial processes operate at single-pass conversion levels of 50 to 60% and with recycling of the unconverted isoprenol, formaldehyde may be present in the recycling stream of unconverted isoprenol, if no steps to purify the stream containing unreacted isoprenol are taken. The recycle stream of unconverted isoprenol has now been found to typically constitute the biggest source of formaldehyde contamination in the reactant stream. The process is generally carried out at partial conversions, for example at conversions of 30 to 70%, preferably 50 to 60%. An unreacted isoprenol stream is separated from the product stream. The unreacted isoprenol stream is recycled, that is, combined with a fresh feed stream comprising isoprenol (a crude isoprenol stream) to provide the reactant stream. The unreacted isoprenol stream comprises isoprenol as a main constituent, but may also comprise prenal, isoprenal, isoamyl alcohol, isovaleraldehyde, isovaleric acid, prenol, formaldehyde. It can also contain traces of other C₃ and C₂ aldehydes and acids.

Prenal may be contained in the isoprenol stream sent to the reactor, that is as an impurity originating from the isoprenol manufacture step. The isoprenol stream may further contain traces of ammonia, and/or C₅-oxygenates other than prenal besides formaldehyde and/or prenal. All the prenal and/or other impurities that cannot be separated in the purification step following the isoprenol synthesis ends up in the reactant stream.

Since the double bond isomerization of isoprenol is an equilibrium reaction, conversion is necessarily incomplete. For economic operation of the process, the unconverted isoprenol has to be removed and recycled. Recycling of isoprenol may therefore inadvertently (re)introduce formaldehyde into the isomerization step if no steps to purify the stream containing unreacted isoprenol are taken.

Reducing the concentration of aldehydes, preferably formaldehyde and/or prenal in the reactant stream or reducing the weight ratio of aldehydes, preferably formaldehyde and/or prenal to isoprenol in the reactant stream can be accomplished in several different ways.

In a preferred embodiment, the process includes separating an unreacted isoprenol stream from the prenol containing product stream, optionally removing at least some aldehydes, preferably some formaldehyde and/or prenal from the unreacted isoprenol stream, followed by combining the unreacted isoprenol stream with a fresh isoprenol stream to form the reactant stream.

In another preferred embodiment, the process includes separating an unreacted isoprenol stream from the prenol containing product stream, combining the unreacted isoprenol stream with a crude isoprenol stream containing isoprenol, water and aldehydes, and removing aldehydes, preferably water and aldehydes from the combined stream to form the reactant stream.

As mentioned above, the crude isoprenol stream is generally the product stream of an isoprenol production process from which unreacted isobutylene has been removed. This means that aldehyde removal (preferable formaldehyde and/or prenal removal, in particular formaldehyde removal) is accomplished in the purification unit following the isoprenol synthesis. A preferred method of recovering formaldehyde from a crude isoprenol stream to which an unreacted isoprenol stream is admixed, is described in more detail below.

Aldehydes, preferably formaldehyde and/or prenal, may be removed from isoprenol streams by a conventional separating method such as distillation, selective adsorption and/or selective reaction.

Removal of aldehydes, preferably formaldehyde and/or prenal, by distillation can involve the use of a single distillation column or a train of distillation columns. The towers and columns used may be conventional distillation columns. Suitable types of distillation columns include packed columns, such as columns with random packing or structured packing, plate columns (i.e., tray columns), and mixed columns comprising both packings and trays.

Suitable plate columns may comprise internals over which the liquid phase flows. Suitable internals include sieve trays, bubble cap trays, valve trays, tunnel trays and Thormann® trays, in particular bubble cap trays, valve trays tunnel trays and Thormann® trays. Random packed columns may be filled with a variety of shaped bodies. Heat and mass transfer are improved by enlarging the surface area by means of shaped bodies, which usually have a size in the range of 25 to 80 mm. Suitable shaped bodies include Raschig rings (hollow cylinders), Lessing rings, Pall rings, Hiflow rings and Intalox saddles. The packing materials may be provided in the column in a regular or irregular manner (as bulk material, i.e., loosely filled). Suitable materials include glass, ceramics, metal and plastics.

Structured packings are an advancement of regular packings and have a regularly shaped structure. This allows for the reduction of gas flow pressure loss. Suitable types of structured packings include fabric and metal sheet packings.

Removal of aldehydes, preferably formaldehyde and/or prenal, by selective adsorption involves contacting the stream with an adsorbent that exhibits selectivity for low molecular weight aldehydes, especially formaldehyde and/or prenal. Useful adsorbent materials should deliver high selectivity and high adsorption capacity. An additional and critically important requirement is that the adsorbent material should not catalyze or participate in chemical reactions that might lower the recovery of the (iso)prenal and/or render the adsorbent inactive. Adsorbents include ion exchange resins, mesoporous solids, activated carbons, and zeolites.

Removal of aldehydes, preferably formaldehyde and/or prenal by selective reaction involves exposing the stream to reaction conditions under which aldehydes, preferably formaldehyde and/or prenal are (is) selectively reacted to products that are less prone to catalyst deactivation and clogging or to products that can be separated from the stream more easily than aldehydes, preferably formaldehyde and/or prenal.

Preferably, removal of aldehydes, preferably formaldehyde and/or prenal from a stream comprising isoprenol is conducted by distillation, selective adsorption and/or selective reaction, in particular by purification process involving the pressure-swing distillation.

The above described applies for the reducing the concentration of aldehydes other than formaldehyde or prenal and/or of other impurities in the reactant stream as well as reducing the weight ratio of aldehydes other than formaldehyde or prenal to isoprenol.

Generally, the isomerization of isoprenol to 3-methyl-2-buten-1-ol (prenol) may be carried out over a supported noble metal, preferably in the presence of hydrogen.

A preferred catalyst is a fixed bed catalyst containing palladium and selenium or tellurium or a mixture of selenium and tellurium supported on silicium dioxide (also: silicon dioxide).

The catalyst contains 0.1 to 2.0 wt.-% of palladium and 0.01 to 0.2 wt.-% of selenium, tellurium or a mixture of selenium and tellurium, based on the total weight of the catalyst.

The BET surface area is, for example, in the range of 100 to 150 m²/g, in particular in the range of 110 to 130 m²/g. The BET surface area is determined by N₂ adsorption according to DIN 66131.

The pore volume in the pore diameter range from 3 nm to 300 μm is preferably 0.8 to 0.9 cm³/g, in particular 0.8 to 0.85 cm³/g. Thereby, 80 to 95%, preferably 85 to 93% of this pore volume is in the pore diameter range of 10 to 100 nm. The pore volume is determined by Hg Porosimetry.

Preferably, the catalyst contains 0.2 to 0.8 wt.-%, in particular 0.4 to 0.6 wt.-% of palladium. Preferably, the catalyst contains 0.02 to 0.08, in particular 0.04 to 0.06 wt.-% selenium, tellurium or a mixture of selenium and tellurium, preferably selenium. In addition to the active components mentioned, other metals may be present on the catalyst in small amounts. Preferably, only palladium, selenium and/or tellurium, in particular only palladium and selenium, are present on the silica support.

The described isomerization of isoprenol to prenol on a fixed-bed catalyst is also described in EP-A 841 090, to which express reference is made.

The isomerization is carried out at a temperature in the range of 50 to 150° C., preferably in the range of 60 to 130° C., more preferably in the range of 70 to 120° C. to produce a reaction mixture of prenol and isoprenol. The isoprenol can be recycled. Further details are provided in WO 2008/037693.

Generally, a regeneration cycle is performed periodically, to remove accumulated coke from the catalyst. The regeneration cycle can be initiated when the pressure drop increased above a threshold value, or at arbitrary time intervals, for example once a week. A regeneration cycle consists of sending diluted air or air for a defined period of time, for example 6 to 24 h, over the reactor while increasing the salt bath temperature, for example 400 to 450° C., to allow coke combustion.

The unreacted isoprenol from the isoprenol isomerization process may be used, i.e., recycled for the isoprenol isomerization.

The selective recovery of aldehydes, especially formaldehyde and/or prenal from the product stream containing an aqueous alcoholic solution is extremely difficult. For example, in case of formaldehyde, this difficulty arises from the fact that monomeric formaldehyde (as well as polymeric formaldehyde) forms both hydrates with water and hemiformals with alcohols such as isoprenol, which is the reactant of the isoprenol isomerization and may still remain in the product stream as unreacted reactant. The hydrates and hemiformals of varying formaldehyde polymerization degree have intermingling boiling points. The stability of and the equilibrium between hydrates and hemiformals is temperature-dependent. Formals formed in an upper region of a distillation tower may decompose in the hotter bottom of the tower, which adds additional complexity to the separation task.

It has been recently reported that the formaldehyde can be separated virtually completely from isoprenol in a distillation train involving a first distillation at a lower temperature at which the equilibrium is shifted towards the hemiformal of formaldehyde and isoprenol, so that essentially all formaldehyde remains in the bottoms of the distillation, and a second distillation at a higher temperature at which the hemiformal is cleaved to formaldehyde and isoprenol, so that the formaldehyde can be easily separated from the isoprenol. See M. Dyga, A. Keller, and H. Hasse, Industrial & Engineering Chemistry Research 2021, 60, 11, 4471-4483.

In an embodiment, the unreacted isoprenol stream is combined with a crude isoprenol stream containing isoprenol, water and aldehydes, preferably formaldehyde and/or prenal; and removing aldehydes, such as formaldehyde and/or prenal, preferably water and aldehydes, in particular water and formaldehyde and/or prenal, from the combined stream comprises:
(i) directing the combined stream to a first low-boiler separation tower operated at a pressure of 1.5 bara or lower, to obtain a first bottoms stream containing isoprenol and aldehydes, preferably prenal and/or formaldehyde, and a first distillate stream containing water and low-boilers;
(ii) directing the first bottoms stream to a second low-boiler separation tower operated at a pressure of 2 bara or higher, to obtain a second distillate stream containing aqueous aldehydes, preferably prenal and/or formaldehyde, and a second bottoms stream containing isoprenol; and
(iii) directing the second bottoms stream to a finishing tower to obtain a bottoms stream containing high-boilers, and the reactant stream as a distillate stream.

In order to permit a first distillation at a temperature below the isoprenol-aldehyde dissociation temperature of the respective aldehyde(s) present, for example for formaldehyde the isoprenol-formaldehyde dissociation temperature and a second distillation at a temperature above the isoprenol-aldehyde dissociation temperature, like the isoprenol-formaldehyde dissociation temperature, the invention envisages two low-boiler separation towers operated at different pressures. Hence, at the relatively low pressure prevailing in the first low-boiler separation tower, a first distillate containing water and low-boilers essentially free of aldehydes, preferably formaldehyde and/or prenal, is obtained. At the relatively high pressure prevailing in the second low-boiler separation tower, a virtually all aldehydes, preferably all formaldehyde and/or prenal, is separated from the isoprenol. The process of the invention thus allows for obtaining isoprenol essentially free of aldehydes, preferably formaldehyde and/or prenal.

The term “essentially free of aldehydes, preferably formaldehyde and/or prenal” is understood to indicate the absence of significant amounts of aldehydes, preferably formaldehyde and/or prenal in the obtained isoprenol. Thus, the obtained isoprenol preferably comprises less than 0.2 wt.-%, in particular less than 0.15 wt.-%, or less than 0.1 wt.-%, based on the total weight of the obtained isoprenol, of aldehydes, preferably formaldehyde and/or prenal.

Preferably, the crude isoprenol stream is a liquid stream. The liquid stream can be a single-phase liquid stream or a two-phase liquid stream.

The crude isoprenol is directed to a first low-boiler separation tower operated at a pressure of 1.5 bara or lower. Any higher pressure of the crude isoprenol stream is preferably released before the same is directed to the first low-boiler separation tower. The crude isoprenol stream is preferably fed to the first low-boiler separation tower as a side stream, defining a rectifying section above the location of the feed and a stripping section below the location of the feed.

In the first low-boiler separation tower, a first bottoms stream containing isoprenol and aldehydes, preferably formaldehyde and/or prenal, and a first distillate stream containing water and low-boilers are obtained. The term “low-boilers” is understood to refer to organic compounds (other than aldehydes, especially formaldehyde and/or prenal) having a boiling point lower than that of isoprenol, hence a boiling point of lower than about 130° C., at atmospheric pressure. The most common low-boilers are methanol and/or isoprenyl formate formed as by-products during the process.

In a preferred embodiment, the first low-boiler separation tower is operated at a pressure of 1.2 bara or lower, preferably 0.5 bara or lower. The bottoms temperature of the first low-boiler separation tower is preferably in the range of 80 to 135° C., more preferably 90 to 115° C., most preferably 95 to 105° C. The temperature at the top of the first low-boiler separation tower is preferably in the range of 45 to 105° C., more preferably 55 to 80° C.

In a particularly preferred embodiment, the first low-boiler separation tower is operated at a pressure in the range of 0.2 to 0.5 bara, a bottoms temperature in the range of 90 to 115° C. and a temperature at the top in the range of 55 to 80° C.

The first low-boiler separation tower preferably has from 15 to 65 theoretical plates, more preferably from 25 to 40 theoretical plates. In particular, the stripping section of the first low-boiler separation tower preferably has 10 to 25 theoretical plates. The rectifying section of the first low-boiler separation tower preferably has 5 to 40 theoretical plates.

The first bottoms stream preferably comprises 75 to 95 wt.-% of isoprenol, more preferably 80 to 90 wt.-%, based on the total weight of the first bottom stream.

The first distillate is typically withdrawn at the top of the first low-boiler separation tower in gaseous form and condensed to obtain a liquid two-phase stream. The two-phase stream is preferably allowed to phase-separate in a separating vessel to obtain an aqueous phase and an organic phase. The aqueous phase is preferably passed to a wastewater stripping column described below. The organic phase is preferably partially returned to the top of the first low-boiler separation tower as a reflux stream. Another part of the organic phase is preferably discarded from the process to avoid the accumulation of water-insoluble low-boilers in the first low-boiler separation tower.

In a preferred embodiment, at least part of the first distillate stream is directed to a wastewater stripping column to separate low-boilers and entrained isoprenol from water. Preferably, the part of the first distillate stream directed to the wastewater stripping column is an aqueous phase obtained by condensation and phase separation of the first distillate stream, as discussed above.

In the wastewater stripping column, low-boilers are obtained as the low-boiler distillate stream, and wastewater is obtained as a bottoms stream. Both the low-boiler distillate stream and the wastewater bottoms stream are removed from the process, and each stream may be directed to further processing.

Moreover, isoprenol is preferably obtained as a side stream in the wastewater stripping column. The isoprenol side stream is typically a two-phase stream and preferably comprises 15 to 40 wt.-% of isoprenol, more preferably 25 to 35 wt.-%, based on the total weight of the isoprenol side stream. The isoprenol side stream is preferably recycled to the first low-boiler separation tower.

The low-boiler distillate stream preferably comprises 75 to 95 wt.-% of low-boilers, more preferably 80 to 85 wt.-%, based on the total weight of the low-boiler distillate stream. The wastewater bottoms stream preferably comprises less than 1.2 wt.-% of organic matter, more preferably less than 0.6 wt.-%, based on the total weight of the wastewater bottoms stream. The wastewater bottoms stream typically comprises aldehydes, preferably formaldehyde and/or prenal in a concentration of 0.05 to 1.5 wt.-% of aldehydes, preferably formaldehyde and/or prenal, such as 0.3 to 0.9 wt.-%, based on the total weight of the wastewater bottoms stream.

The wastewater stripping column is preferably operated at a pressure of 1.5 bara or lower, preferably 1.1 bara or lower. The bottoms temperature of the wastewater stripping column is preferably in the range of 95 to 110° C., more preferably 97 to 103° C. The temperature at the top of the wastewater stripping column is preferably in the range of 65 to 100° C., more preferably 75 to 85° C.

In a particularly preferred embodiment, the wastewater stripping column is operated at a pressure in the range of 0.95 to 1.1 bara, a bottoms temperature in the range of 97 to 103° C. and a temperature at the top in the range of 75 to 85° C.

The wastewater stripping column preferably has from 6 to 30 theoretical plates, more preferably from 10 to 20 theoretical plates.

The first bottoms stream obtained in the first low-boiler separation tower is directed to a second low-boiler separation tower operated at a pressure of 2 bara or higher. The first bottoms stream is preferably fed to the second low-boiler separation tower as a side stream, defining a rectifying section above the location of the feed and a stripping section below the location of the feed.

In the second low-boiler separation tower, a second distillate stream containing or consisting essentially of aqueous aldehydes, preferably formaldehyde and/or prenal, and a second bottoms stream containing isoprenol are obtained. The second bottom stream further comprises high-boilers. The term “high-boilers” is understood to refer to organic compounds having a boiling point higher than that of isoprenol, i.e., higher than about 130° C., at atmospheric pressure.

In a preferred embodiment, the second low-boiler separation tower is operated at a pressure of 2.5 bara or higher, preferably 2.8 bara or higher, most preferably 2.9 bara or higher. The bottoms temperature of the second low-boiler separation tower is preferably in the range of 160 to 200° C., more preferably 170 to 185° C., most preferably 175 to 180° C. The temperature at the top of the second low-boiler separation tower is preferably in the range of 115 to 160° C., more preferably 125 to 145° C.

In a particularly preferred embodiment, the second low-boiler separation tower is operated at a pressure in the range of 2.9 to 3.5 bara, a bottoms temperature in the range of 175 to 180° C. and a temperature at the top in the range of 130 to 140° C.

The second low-boiler separation tower preferably has from 20 to 60, more preferably from 35 to 60 theoretical plates. In particular, the stripping section of the first low-boiler separation tower preferably has 25 to 45 theoretical plates. The rectifying section of the first low-boiler separation tower preferably has 7 to 20 theoretical plates.

At the top of the second low-boiler separation tower, an offgas is typically obtained. The offgas primarily comprises nitrogen and may comprise traces of isoprenol, formic acid, water, aldehydes, preferably formaldehyde, prenal and/or decomposition gases.

The second bottoms stream preferably comprises 82 to 96 wt.-% of isoprenol, more preferably 87 to 91 wt.-%. The relatively high pressure of the second low-boiler separation tower allows for a high degree of separation of aldehydes, preferably formaldehyde and/or prenal, and isoprenol. Thus, the second bottoms stream preferably comprises at most 0.5 wt.-%, more preferably at most 0.1 wt.-%, even more preferably at most 0.008 wt.-% of aldehydes, preferably formaldehyde and/or prenal, based on the total weight of the second bottoms stream.

The second distillate stream is an aqueous stream, which preferably comprises 25 to 60 wt.-%, more preferably 40 to 50 wt.-%, in particular 45 to 50 wt.-%, based on the total weight of the second distillate stream, of aldehydes, preferably formaldehyde and/or prenal. The second distillate stream preferably comprises at most 15 wt.-% of isoprenol, more preferably at most 5 wt.-%, based on the total weight of the second distillate stream, of isoprenol.

Owing to the broad condensation curve of the vapor emerging at the top of the second low-boiler separation tower, it is advantageous to use a condenser with liquid recycling. The direct condensation in a quench with liquid circulation is particularly advantageous. Hence, in a preferred embodiment of the process, a quench section is provided downstream, in vapor flow direction, of the rectifying section of the second low-boiler separation tower. The term “vapor flow direction” relates to the direction of the flow of gaseous components in the separation tower, i.e., upwards, towards the top of the tower. The quench section is preferably provided within the second low-boiler separation tower above the rectifying section.

The direct condensation in a quench also mitigates fouling caused by various condensation and polymerization mechanisms of aldehydes, for example formaldehyde that may occur at spots of high local aldehyde concentrations, like local formaldehyde concentrations. To avoid the risk of fouling in the second low-boiler separation tower and downstream processes, in particular in the offgas of the second low-boiler separation tower, the concentration of aldehydes, preferably formaldehyde and/or prenal, in the second distillate is preferably no higher than 60 wt.-%, more preferably no higher than 55 wt.-% and in particular no higher than 50 wt.-%, based on the total weight of the second distillate stream.

At the lower end of the quench section, an aqueous liquid is collected. When the quench section is provided within the second low-boiler separation tower, the aqueous liquid may be collected, e.g., at a collecting tray above the rectifying section and beneath of the quench section.

The aqueous liquid is partially circulated into the quench section through a circulation line and partially withdrawn as the second distillate. Suitably, the part of the aqueous liquid circulated into the quench section is circulated into the top of the quench section. Circulation of the aqueous liquid is typically achieved by use of a pump.

The circulation of a part of the aqueous liquid into the quench section allows for cooling of vapors rising through the quench section, and absorption of aldehydes, preferably formaldehyde and/or prenal from the vapors into the aqueous liquid. Thus, aldehydes, preferably formaldehyde and/or prenal, may be quenched from the vapors rising through the quench section.

Further, the aqueous liquid is partially returned to the rectifying section of the second low-boiler separation tower as a reflux stream. This may be accomplished by a reflux line, or aqueous liquid may be partially returned to the rectifying section as overflow from a collecting tray beneath the quench section.

The mass flow ratio of the reflux stream to the second distillate is preferably in the range of 2:1 to 10:1, more preferably in the range of 3:1 to 7:1. In a preferred embodiment, the aqueous liquid is cooled before being circulated into the quench section. Preferably, the part of the aqueous liquid withdrawn as the second distillate is a partial stream of the cooled aqueous liquid.

The temperature of the aqueous liquid collected at the lower end of the quench section is preferably in the range of 80 to 140° C., more preferably 125 to 135° C. The temperature of the cooled aqueous liquid circulated into the quench section is preferably 10 to 80° C. below the temperature of the aqueous liquid collected at the lower end of the quench section. This allows for an energetically favorable process.

The hot aqueous liquid withdrawn at the lower end of the quench section lends itself to heat-integration. In a suitable embodiment, it is heat-exchanged with the stream of crude isoprenol flowing into the first low-boiler separation tower before being circulated into the quench section.

In one embodiment, a scrubbing section is provided downstream, in vapor flow direction, of the quench section and water is introduced at the top of the scrubbing section. Preferably, the scrubbing section is provided within the second low-boiler separation tower above the quench section. The scrubbing section allows for maintaining the aldehydes, preferably formaldehyde and/or prenal, concentration in the second distillate below the critical concentrations described above and thus to avoid depositions for example paraformaldehyde deposition in, e.g., offgas lines.

The mass flow ratio of the water introduced at the top of the scrubbing section to the first bottoms stream obtained in the first low-boiler separation tower is typically in the range of 0.01:1 to 0.06:1 more preferably in the range of 0.015:1 to 0.03:1.

The second bottoms stream is directed to a finishing tower, in which pure isoprenol is obtained as a distillate stream. High-boilers are withdrawn via a bottoms stream. As the second bottoms stream comprises essentially no aldehydes, preferably no formaldehyde and/or prenal, the separation task of the finishing tower is significantly less complex than in cases where formaldehyde separation is less efficient in the low-boiler separation section.

The term “essentially no aldehydes, preferably no formaldehyde and/or prenal” is understood to indicate the absence of significant amounts of aldehydes, preferably formaldehyde and/or prenal in the obtained isoprenol. Thus, the obtained isoprenol preferably comprises less than 0.05 wt.-%, preferably less than 0.01 wt.-%, based on the total weight of the second bottoms stream, of aldehydes, preferably formaldehyde and/or prenal.

The pure isoprenol distillate stream preferably at least 97.0 wt.-% of isoprenol, more preferably 98.0 wt.-%, such as 98.1 to 99.5 wt.-%, based on the total weight of the pure isoprenol distillate stream. The high-boiler bottoms stream preferably comprises 90 to 99.9 wt.-% of high-boilers, more preferably 99 to 99.8 wt.-%, based on the total weight of the high-boiler bottoms stream. Preferably, the high-boiler bottoms comprises stream less than 0.2 wt.-% of aldehydes, preferably formaldehyde and/or prenal, such as less than 0.05 wt.-%, based on the total weight of the high-boiler bottoms stream, of aldehydes, preferably formaldehyde, and/or prenal.

In a preferred embodiment, the finishing tower is operated at a pressure of 0.5 bara or lower, preferably 0.25 bara or lower. The bottoms temperature of the first low-boiler separation tower is preferably in the range of 130 to 190° C., more preferably 150 to 170° C. The temperature at the top of the finishing tower is preferably in the range of 60 to 90° C., more preferably 65 to 85° C.

In a particularly preferred embodiment, the finishing tower is operated at a pressure in the range of 0.05 to 0.2 bara, a bottoms temperature in the range of 150 to 170° C. and a temperature at the top in the range of 65 to 85° C.

The finishing tower preferably has from 6 to 40 theoretical plates, more preferably from 10 to 20 theoretical plates.

### Oxidizing Prenol to Prenal

The prenol obtained as described above may be oxidized so as to obtain prenal by bringing a reactant stream comprising prenol into contact with at least one oxidant and at least one oxidation catalyst, preferably in the presence of a liquid phase.

Suitable oxidants include hydrogen peroxide and oxygen, in particular oxygen.

The oxidation is preferably carried out in the presence of a liquid phase and with oxygen as the oxidant. The liquid phase preferably comprises at least 25 wt.-% of water, more preferably at least 50 wt.-% of water or at least 70 wt.-% of water, based on the total weight of the liquid phase, determined at a temperature of 20° C. and a pressure of 1 bar. It has been found that these conditions allow for a simple and efficient process for preparing prenal from prenol.

The oxidation is typically carried out in the presence of at least one oxidation catalyst selected from the group consisting of platinum, palladium and gold. Preferably, the at least one oxidation catalyst comprises platinum. In a preferred embodiment, the at least one oxidation catalyst is a supported catalyst.

The oxidation is suitably carried out at a temperature of 20° C. to 100° C., preferably at a temperature of 20° C. to 70° C. The oxidation is suitably carried out under a partial pressure of oxygen between 0.2 and 8 bar.

### Further Conversion to Citral

The prenol obtained as described above may also be useful in the preparation of citral. Citral is a mixture of the isomeric compounds neral and geranial.

3,7-dimethyl-octa-2,6-dienal (citral) can be prepared by obtaining prenol by a process as described above, further comprising the steps of condensing the prenol with prenal to obtain diprenyl acetal of prenal; and subjecting the diprenyl acetal of prenal to cleaving conditions to obtain citral via prenyl (3-methyl-butadienyl) ether and 2,4,4-trimethyl-3-formyl-1,5-hexadiene.

The prenol obtained as described above, preferably the product stream comprising prenol may be used as feed to step of condensing the prenol with prenal to obtain diprenyl acetal of prenal.

More details with respect to the citral preparation using the prenol obtained by a process as described above is described below.

Recycling Stream from the Isomerization of Isoprenol to Prenol

The unreacted isoprenol from the isomerization of isoprenol to prenol, which is conducted according to the invention, may be used, i.e., directed as feed to oxidative dehydrogenation step of isoprenol to obtain a stream comprising prenal and/or isoprenal.

Oxidative dehydrogenation of isoprenol typically comprises bringing a reactant stream, in particular a gaseous reactant stream, comprising isoprenol into contact with at least one heterogeneous oxidative dehydrogenation catalyst, in particular at least one silver-containing heterogeneous oxidative dehydrogenation catalyst, in the presence of molecular oxygen. The at least one heterogeneous catalyst may consist of an inert support having a smooth surface having an active layer of silver. Alternatively, massive (full-metal) silver bodies may be used.

More details with respect to the recycling the unreacted isoprenol from the isomerization of isoprenol to prenol as feed to oxidative dehydrogenation step of isoprenol is described below.

### Second Aspect of the Invention: Process for Preparing Citral

It will be understood that the definitions and embodiments defined for a process for the preparation of prenol mutatis mutandis apply to a process for preparing citral.

### Reacting a Formaldehyde Source and Isobutylene to Obtain Isoprenol

Prenol useful as a starting material for the invention may be obtained by reacting at least one formaldehyde source and isobutylene to obtain 3-methylbut-3-en-1-ol (isoprenol) in a reactor, in general at elevated temperature and pressure, and subjecting the obtained isoprenol to isomerization.

In one embodiment, the isoprenol is obtained by introducing, preferably by mixing and injecting, at least one formaldehyde source and isobutylene into a reactor, preferably through at least one nozzle, and reacting the at least one formaldehyde source and isobutylene under supercritical conditions. In order to achieve supercritical conditions, formaldehyde and isobutylene are preferably reacted at a temperature of at least 220° C., for example in the range of 220 to 290° C., and an absolute pressure of at least 200 bara. The reaction of isobutene and formaldehyde may be carried out without a catalyst as well as in the presence of at least one catalyst. The reaction of isobutene source and formaldehyde may also be carried out in the presence of one or more auxiliary chemicals such as ammonia and/or hexamethylenetetramine (urotropin). Conducting this reaction in the presence of such auxiliary chemicals, especially ammonia and/or urotropin, has been described, e.g., in DE 1279014 B.

For example, the presence of a Bronsted acid compound, such as a Bronsted acid catalyst, may induce the elimination of water from isoprenol, yielding isoprene and water. It is thus preferable that the process of the invention be performed essentially free of compounds inducing further reactions of isoprenol, such as Bronsted acid compounds. Of course, this does not relate to the starting materials and product of the process.

The process is thus preferably performed in the presence of less than 0.5 mol-%, preferably less than 0.1 mol-%, more preferably less than 0.01 mol-% and most preferably less than 0.001 mol-% of compounds capable of inducing further reactions of isoprenol, especially Bronsted acid compounds. A Bronsted acid compound is understood to be any compound with a pH value of less than 7, as measured at 25° C. and 1 bar absolute.

In one embodiment, the process is performed in the presence of a Bronsted base compound. The Bronsted base compound is useful for neutralizing acids that may be present in the starting materials or may be formed in side reactions during the process.

Examples of suitable Bronsted base compounds are hydroxides, carbonates and bicarbonates of alkali metals and alkaline earth metals, ammonia or organic amines, and salts of acids which are weaker than formic acid. It is particularly advantageous to use ammonia or organic amines, such as ethylamine, trimethylamine, hexamethylenetetramine (urotropin), aniline, pyridine, or piperidine. Substances exhibiting a buffer action, such as hexamethylenetetramine (urotropin), are particularly suitable.

The Bronsted base compound is favorably present in amounts of 0.001 to 10 wt.-%, particularly 0.01 to 1 wt.-%, based on the total weight of reaction mixture. It is advantageous to use weak bases and, in order to avoid secondary reactions which may take place in a strongly alkaline range, to ensure that the starting mixture of the reactants, after the Bronsted base compound has been added, has a pH value of from 7 to 11, advantageously from 7 to 10, particularly from 7.5 to 9, as determined at 20° C. and 1 bar absolute. If anhydrous starting materials are used, the pH value of the starting mixture is determined after dilution with an equal amount by weight of water.

The Bronsted base compound may be fed into the reactor at one or more suitable locations. When the formaldehyde source is an aqueous formaldehyde solution, the Bronsted base compound is preferably fed into the reactor dissolved in the aqueous formaldehyde solution. In another embodiment, the Bronsted base compound is separately fed into the reactor as an aqueous solution.

The at least one formaldehyde source and isobutylene are preferably introduced into the reactor in a manner which allows for mixing of the reactants so as to obtain an intimate mixture. Introduction methods include injecting, splashing, stirring in and/or spraying into the reactor. Preferably, the at least one formaldehyde source and isobutylene are injected or sprayed into the reactor through at least one nozzle.

Formaldehyde may be provided as a liquid, for example as a solution of paraformaldehyde in methanol. Preferably, the at least one formaldehyde source comprises or is an aqueous formaldehyde solution.

While initial rapid and intense mixing of reactants is desirable, it may be advantageous to continue and complete the reaction under conditions of limited back-mixing. Thus, the reaction mixture may be passed into a post-reaction chamber disposed after the reactor or in a lower portion of the reactor. In the post-reaction chamber, back-mixing is limited.

In one embodiment, the reactor comprises an upper portion and a lower portion. Introduction of the reactants, in particular by injecting and mixing of the reactants, occurs in a mixing chamber of the reactor disposed in the upper portion, and a fluid comprising formaldehyde and/or isobutylene and/or isoprenol is passed from the mixing chamber into a post-reaction chamber disposed in the lower portion.

In one embodiment, reacting at least one formaldehyde source and isobutylene comprises introducing, preferably mixing and injecting, the at least one formaldehyde source and isobutylene into an internal loop reactor through at least one nozzle into first conduit(s), the internal loop reactor comprising:
a vertically disposed cylindrical vessel comprising a sidewall;
at least one draft tube having a tube inlet end and a tube outlet end, arranged vertically within the vessel, the draft tube(s) being arranged concentrically to the nozzle(s), and having an inner surface and an outer surface, wherein the draft tube(s) provide(s) the first conduit(s) within the draft tube(s), and a second conduit outside of the draft tube(s) and within the sidewall, the first conduit(s) being in fluid communication with the second conduit;
reactor fluid outlet means;
   wherein the inner surface of the draft tube(s) convexly curves so that the first conduit(s) exhibit(s) an annular constriction of the cross-section between the tube inlet end and the tube outlet end; wherein the constriction is located closer to the tube inlet end; wherein the convex curvature of the inner surface of the draft tube(s) extends over at least 70%, preferably at least 80%, most preferably at least 90% of the length of the draft tube; and
   wherein the outer surface of the draft tube(s) convexly curves so that the draft tube(s) exhibit(s) a circumferential protuberance between the tube inlet end and the tube outlet end, which circumferential protuberance is preferably located closer to the tube outlet end; wherein the convex curvature of the outer surface of the draft tube(s) extends over at least 70%, preferably at least 80%, most preferably at least 90% of the length of the draft tube; and
   wherein the edges of the draft tube(s) are rounded so that the at least one formaldehyde source and isobutylene introduced through the nozzles travel generally downward in the first conduit(s) to obtain a reacted fluid, the reacted fluid is then diverted in the opposite direction so as to travel through the second conduit and is subsequently back-mixed with the introduced fluid.

In an embodiment, the configuration of the draft tube(s) allows control of the boundary layer flowing over the edge of the draft tube. When the angle of attack of a flow with respect to a solid body reaches a certain limit, the adverse pressure gradient may become too large for the flow to negotiate it. At this point, the flow may separate from the upper surface of the body, resulting in a condition commonly known as stall. The configuration may allow for decreased flow separation or a delay in flow separation, respectively. Decreased flow separation may allow for reduced liquid friction and thus may lead to a lower pressure drop along the streamline of the recirculating flow, which in turn results in a higher circulation ratio of the configuration. The curved shape of the inner surface of the draft tube wall may guide the fluid through the draft tube in an optimized manner, comparable to the flow of fluid over an airfoil.

The inner surface of the draft tube may curve in the longitudinal direction of the draft tube, or in other words may have a convex shape, so that the first conduit may exhibit a minimum cross-section between the tube inlet end and the tube outlet end. This means that the cross-section of the first conduit may decrease from the cross-section at the tube inlet end to a minimum cross-section and may increase from the minimum cross-section to the cross-section at the tube outlet end.

The draft tube may have a curved, approximately conical section between the tube inlet end and the constriction, being wide at the inlet end and narrower at the constriction. At least some of the fluid flowing downstream through the draft tube may be deflected so as to flow along the inner surface of the draft tube until the draft tube ends. The flow through the tube may predominantly remain attached, thus generating less pressure loss. In the vicinity of the constriction, the fluid flowing downstream through the draft tube may be accelerated. Between the constriction and the tube outlet end, the cross-sectional area of the draft tube may widen again. Consequently, the area variation, in conjunction with mass conservation, may force the velocity through the larger area to be slower than through the smaller one, accompanied by a conversion of the dynamic pressure into static pressure. Acceleration of the fluid flowing downstream through the draft tube in the vicinity of the constriction may add a radial velocity component to the flow, increasing the mixing between circulating flow and injected flow. By avoiding flow separation in this case, no significant pressure loss occurs.

In a preferred embodiment, the nozzles are two-component nozzles. It is especially preferable that a two-component nozzle is designed so as to provide an annular jet of isobutylene around a central jet of the at least one formaldehyde source, and that the velocities upon introduction, for example the injection velocities or spraying velocities, of these two jets are different. In this embodiment, the jet of isobutylene has a large shear surface towards both the central jet of the at least one formaldehyde source and the reaction mixture in the reactor, allowing for favorable fast mixing of the reactants.

In a preferred embodiment, the loop reactor comprises deflector means arranged between the nozzle and the draft tube, the deflector means being suitable for deflecting fluid travelling in the second conduit in the opposite direction.

The deflector means suitably comprise a surface which is concave relative to the end of the draft tube which defines the tube inlet end. In a preferred embodiment, the deflector means have a partial toroidal surface. It is especially preferred that the deflector means are provided in the shape of the upper portion of a ring torus bisected in a plane parallel to the toroidal direction. This shape allows for an especially efficient deflection of the fluid travelling in the second conduit. The deflector means may allow for a stabilization of the introduced, for example injected or sprayed fluid stream. This is especially relevant when the flow rate of the fluid travelling in the second conduit is not uniform across the cross section of the reactor, which may lead to an eccentricity of the introduced fluid stream. Such an eccentricity may cause a decrease in circulation ratio if left unattended.

When the first conduit is downcomer conduit and the second conduit is a riser conduit, it is preferred that the shape of the deflector means constitutes the upper portion of a ring torus bisected in a plane parallel to the toroidal direction, wherein the ring torus is bisected at least 50% of its height, such as at least 55% or 65% of its height. Thus, the upper portion of the ring torus is the same size or smaller than the lower portion of the ring torus. In another preferred embodiment, the shape of the deflector means constitutes the upper portion of a ring torus bisected in a plane parallel to the toroidal direction wherein the ring torus is bisected at most 85% of its height, for example 80% of its height. In these ranges, the entry of the deflector means is angled especially suitable for fluid deflection.

Further details regarding aforementioned embodiments concerning loop reactor may be found in WO 2023/104863, which herewith is incorporated by reference in its entirety.

High temperatures are required to obtain a high isoprenol yield in the reaction of formaldehyde with isobutylene. Effective removal of the heat is critical for the product quality and process safety. The heat removed from the isoprenol is used for raising the temperature of isobutylene before it enters the reactor. The stream of the hot isoprenol contains sensible heat from the chemical reaction. The sensible heat is potentially reclaimable energy that can be reused.

Advantageously, reacting at least one formaldehyde source and isobutylene preferably comprises heat-exchanging a stream of hot isoprenol withdrawn from the reactor with a isobutylene stream directed to the reactor; wherein heat-exchanging is performed in one or more shell-and-tube heat exchangers; each of the heat exchangers comprising a plurality of tubes and a shell-side heat exchange passage; wherein the hot isoprenol is directed through the tubes of the heat exchangers; and the isobutylene is guided through the shell-side passage, and in case of more than one heat exchangers at least two of the heat exchangers are connected in series with regard to both the shell-side flow and the tube-side flow.

In a group of the preferred embodiments, the heat-exchanging is performed in one shell-and-tube heat exchanger.

In another group of the preferred embodiments, the heat-exchanging is performed in at least one or more shell-and-tube heat exchangers, wherein the hot isoprenol is directed through the tubes of the heat exchangers; and the isobutylene is guided through the shell-side passage, and in case of at least two of the heat exchangers these are connected in series with regard to both the shell-side flow and the tube-side flow.

Such configurations allow for prolonging operation intervals between maintenance disruptions in such a method. The term “maintenance disruptions” is intended to mean a shutdown of the process that becomes necessary at recurring intervals in order to clear the tubes of the heat exchanger that have been clogged by fouling. An indicator of a necessity of a maintenance disruption is typically when isobutylene leaving the last heat exchanger is insufficiently pre-heated and that even a subsequent heater is hardly able to put in additional external heat into the isobutylene to bring isobutylene to the required temperature before it enters the reactor. One aspect of the invention is that the pre-heating of the isobutylene stream can be maintained for a longer time at levels high enough so that the desired temperature of the isobutylene can easily be reached before the isobutylene enters the reactor.

One particular area prone to fouling in conventional shell-and-tube heat exchangers is the tube area near the tube sheet near the inlet where the tube-side fluid leaves the individual tubes. Excessive fouling in this area can cause clogging of individual tubes and fluid stagnation along the entire length of these tubes. The fluid stagnation generally leads to reduced heat-transfer performance.

As a further consequence of the decreased heat transfer performance caused by fouling, the energy required in a heater to adjust the temperature of the pre-heated isobutylene stream to the desired reaction temperature increases. Consequently, more additional external heat becomes necessary which is detrimental in terms of energy demand and process economy, and often has a negative impact on the carbon dioxide footprint of the product.

By using two or more heat exchangers, the impact of fouling in individual tubes on the overall heat exchange capacity is reduced in comparison to arrangements where only a single heat exchanger is used. As a consequence, the heat transfer rates are maintained at a desired level for longer periods, hence prolonging operation intervals between maintenance disruptions, and the pre-heating of the isobutylene stream requires less additional external heat compared to a plant with a single heat exchanger in an advanced state of fouling.

Further details regarding aforementioned embodiments concerning heat exchangers and energy savings and reducing maintenance intervals may be found in WO 2023/198714 A1, which herewith is incorporated by reference in its entirety.

Removal of Unreacted Formaldehyde from Crude Isoprenol Separation of the isoprenol from unreacted formaldehyde is not a trivial task. This difficulty arises from the fact that monomeric formaldehyde (as well as polymeric formaldehyde) forms both hydrates with water and hemiformals with isoprenol. The hydrates and hemiformals of varying formaldehyde polymerization degree have intermingling boiling points.

It has, however, been found that formaldehyde can be separated virtually completely from isoprenol via distillation at a temperature at which the hemiformal is cleaved to formaldehyde and isoprenol, so that the formaldehyde can be easily separated from the isoprenol. The separated formaldehyde may be recycled into the isoprenol synthesis.

Hence, crude isoprenol may be purified by subjecting a stream of crude isoprenol containing isoprenol, water and formaldehyde, or an isoprenol containing fraction thereof, to distillation in a low-boiler separation tower operated at a pressure of 2 bara or higher, preferably 2.5 bara or higher, to obtain a distillate stream containing aqueous formaldehyde and a bottoms stream containing isoprenol essentially free of formaldehyde.

The term “essentially free of formaldehyde” is understood to indicate the absence of significant amounts of formaldehyde in the obtained pure isoprenol. Thus, the obtained pure isoprenol preferably comprises less than 0.5 wt.-%, more preferably less than 0.1 wt.-% of formaldehyde. The term “crude isoprenol” as used herein indicates a stream comprising isoprenol, water and formaldehyde. Preferably, the crude isoprenol stream comprises 50 to 75 wt.-% of isoprenol, more preferably 60 to 65 wt.-%. Preferably, the crude isoprenol stream comprises 15 to 40 wt.-% of water, more preferably 22 to 35 wt.-%. Preferably, the crude isoprenol stream comprises 1 to 5 wt.-% of formaldehyde, more preferably 2 to 3 wt.-%.

In particular, it has been found that the formaldehyde can be separated virtually completely from isoprenol and concentrated aqueous formaldehyde suitable for recycling into the isoprenol synthesis can be obtained in a distillation train involving a first distillation at a temperature at which the equilibrium is shifted towards the hemiformal of formaldehyde and isoprenol, so that essentially all formaldehyde remains in the bottoms of the distillation, and a second distillation at a temperature at which the hemiformal is cleaved to formaldehyde and isoprenol, so that the formaldehyde can be easily separated from the isoprenol.

In order to permit a first distillation at a temperature below the isoprenol-formaldehyde dissociation temperature and a second distillation at a temperature above the isoprenol-formaldehyde dissociation temperature, two low-boiler separation towers operated at different pressures are envisioned. Hence, at the relatively low pressure prevailing in the first low-boiler separation tower, a first distillate containing water and low-boilers essentially free of formaldehyde is obtained. At the relatively high pressure prevailing in the second low-boiler separation tower, a virtually all formaldehyde is separated from the isoprenol. This process thus allows for obtaining isoprenol essentially free of formaldehyde.

Hence, in a more preferred embodiment, the purification process comprises:
(i) directing the stream of crude isoprenol to a first low-boiler separation tower operated at a pressure of 1.5 bara or lower, to obtain a first bottoms stream containing isoprenol and formaldehyde, and a first distillate stream containing water and low-boilers;
(ii) directing the first bottoms stream to a second low-boiler separation tower operated at a pressure of 2 bara or higher, to obtain a second distillate stream containing aqueous formaldehyde, and a second bottoms stream containing isoprenol; and
(iii) directing the second bottoms stream to a finishing tower to obtain pure isoprenol as a distillate stream, and a bottoms stream containing high-boilers.

The second distillate stream constitutes concentrated aqueous formaldehyde fit for recycle into the isoprenol synthesis.

Suitably, the second low-boiler separation tower is operated at a pressure of 2.5 bara or higher, preferably 2.8 bara or higher, most preferably 2.9 bara or higher. The bottoms temperature of the second low-boiler separation tower is preferably in the range of 160 to 200° C., more preferably 170 to 185° C., most preferably 175 to 180° C. The temperature at the top of the second low-boiler separation tower is preferably in the range of 115 to 160° C., more preferably 125 to 145° C.

In a particularly preferred embodiment, the second low-boiler separation tower is operated at a pressure in the range of 2.9 to 3.5 bara, a bottoms temperature in the range of 175 to 180° C. and a temperature at the top in the range of 130 to 140° C.

Detailed information on the process for recovering isoprenol with low concentration of or essentially free of formaldehyde may be found in WO 2022/189652 A1.

Further details regarding reacting at least one formaldehyde source and isobutylene to obtain isoprenol may be found in WO 2020/049111 A1, which herewith is incorporated by reference in its entirety.

### Isomerizing Isoprenol to Obtain Prenol

The obtained isoprenol may be subjected to catalytic isomerization by bringing a reactant stream comprising isoprenol into contact with at least one heterogeneous isomerization catalyst, so as to obtain prenol.

Isomerization of isoprenol to 3-methyl-2-buten-1-ol (prenol) may be carried out over a supported noble metal, preferably in the presence of hydrogen. A preferred catalyst is a fixed bed catalyst containing palladium and selenium or tellurium or a mixture of selenium and tellurium supported on silicium dioxide (also: silicon dioxide). The isomerization may be carried out at a temperature of 50 to 150° C. to produce a reaction mixture of prenol and isoprenol. The isoprenol can be recycled. Further details, as a preferred embodiment, are provided in WO 2008/037693.

When isoprenol is subjected to catalytic isomerization, it may be favorable to maintain in the reactant stream a weight ratio of formaldehyde to isoprenol of less than 0.04, preferably less than 0.03, in particular less than 0.02, or less than 0.01. In still more preferred embodiments, the weight ratio of formaldehyde to isoprenol is maintained at less than 0.002, or less than 0.001.

It has been found that the presence of formaldehyde in the reactant stream may accelerate catalyst deactivation and/or poisoning.

The weight ratio of formaldehyde to isoprenol in the reactant stream may be maintained at a certain level or less. Reducing the weight ratio of formaldehyde to isoprenol in the reactant stream beyond a certain point, however, reaches a point of rapidly diminishing return. Formaldehyde removal involves additional equipment and operating costs. An economic balance must be taken between the improvement due to reducing the ratio and the cost of maintaining such a ratio. Hence, the weight ratio of formaldehyde to isoprenol is preferably not lower than 0.0005 or, in some instances, not lower than 0.005.

The presence of formaldehyde in the reactant stream is due to two main sources. Formaldehyde may be contained in the fresh feed stream sent to the reactor, that is as an impurity originating from the isoprenol manufacture step. All the formaldehyde that cannot be separated in the purification step following the isoprenol synthesis ends up in the reactant stream.

In addition, formaldehyde is also generated in situ. Part of the isoprenol splits back to isobutene and formaldehyde.

Since the double bond isomerization of isoprenol is an equilibrium reaction, conversion is necessarily incomplete. For economic operation of the process, the unconverted isoprenol has to be removed and recycled. Recycling of isoprenol may inadvertently (re)introduce formaldehyde into the isomerization step if no steps to purify the stream containing unreacted isoprenol are taken.

Reducing the weight ratio of formaldehyde to isoprenol in the reactant stream can be accomplished in several different ways. In an embodiment, formaldehyde is removed from the unreacted isoprenol stream prior to combining the unreacted isoprenol stream with the fresh feed stream.

In an embodiment, the unreacted isoprenol stream is combined with the fresh feed stream and formaldehyde is removed from the combined stream.

Reference is made to the first aspect of the invention with respect to the isomerization of isoprenol to 3-methyl-2-buten-1-ol (prenol). Isoprenol obtained as described above is isomerized to obtain prenol by bringing a reactant stream comprising isoprenol into contact with at least one heterogeneous isomerization catalyst, preferably in the presence of hydrogen, optionally by maintaining or adjusting in the reactant stream a concentration of aldehydes of less than 0.5 wt.-%, preferably less than 0.4 wt.-%, in particular less than 0.3 wt.-%, or less than 0.25 wt.-%, based on the total weight of the reactant stream, wherein further optionally the concentration of aldehydes in the reactant stream is not lower than 10 ppm, preferably not lower than 25 ppm, in particular not lower than 50 ppm, or not lower than 100 ppm, with respect to the total weight of the reactant stream.

Alternatively, it is feasible to mix the unreacted isoprenol stream with an amount of a sufficiently purified fresh feed stream so as to give in the combined stream a desired weight ratio of formaldehyde to isoprenol.

Aldehydes, preferably formaldehyde and/or prenal may be removed from the streams comprising isoprenol by a conventional separating method such as distillation, selective adsorption and or selective reaction, in particular by the purification process involving the pressure-swing distillation as described above.

In an embodiment, the process for preparing prenol is a continuous process. In an embodiment, all steps for preparing prenol are conducted continuously, in particular are conducted as consecutive continuous steps. This may for instance include steps for preparing isoprenol (e.g., from formaldehyde and isobutene) and isomerization to prenol and optional one or more purification steps.

### Providing Prenal

The isoprenol obtained as described above is converted to prenal, involving isomerization and an oxidative dehydrogenation in any order. Thus, it is possible to first isomerize isoprenol to prenol, and subsequently oxidize prenol to prenal; or, to first oxidatively dehydrogenate isoprenol to isoprenal, and optionally isomerize at least part of the isoprenal to prenal.

### Oxidizing Prenol to Prenal

The prenol obtained as described above may be oxidized so as to obtain prenal by bringing a reactant stream comprising prenol into contact with at least one oxidant and at least one oxidation catalyst, preferably in the presence of a liquid phase.

Suitable oxidants include hydrogen peroxide and oxygen, in particular oxygen.

The oxidation is preferably carried out in the presence of a liquid phase and with oxygen as the oxidant. The liquid phase preferably comprises at least 25 wt.-% of water, more preferably at least 50 wt.-% of water or at least 70 wt.-% of water, based on the total weight of the liquid phase, determined at a temperature of 20° C. and a pressure of 1 bar. It has been found that these conditions allow for a simple and efficient process for preparing prenal from prenol.

The oxidation is typically carried out in the presence of at least one oxidation catalyst selected from the group consisting of platinum, palladium and gold. Preferably, the at least one oxidation catalyst comprises platinum. In a preferred embodiment, the at least one oxidation catalyst is a supported catalyst.

The oxidation is suitably carried out at a temperature of 20° C. to 100° C., preferably, 25° C. to 80° C., in particular 30 to 70° C., in particular 35 to 50° C. In another embodiment the oxidation is carried out at a temperature of 20° C. to 70° C. The oxidation is suitably carried out under a partial pressure of oxygen between 0.2 and 8 bar.

Further details of the oxidation reaction may be found in WO 2023/222895 A1, which herewith is incorporated by reference in its entirety.

### Oxidative Deyhdrogenation of Isoprenol

Oxidative dehydrogenation of isoprenol typically comprises bringing a reactant stream, in particular a gaseous reactant stream, comprising isoprenol into contact with at least one heterogeneous oxidative dehydrogenation catalyst, in particular at least one silver-containing heterogeneous oxidative dehydrogenation catalyst, in the presence of molecular oxygen. The at least one heterogeneous catalyst may consist of an inert support having a smooth surface having an active layer of silver. Alternatively, massive (full-metal) silver bodies may be used.

In an embodiment, the non-reacted isoprenol from the isomerization of isoprenol to prenol, which may correspond to the step b), is used as feed to the dehydrogenation step.

Hence, in an embodiment, the process includes separating an unreacted isoprenol stream from a prenol containing product stream which may be obtained in step b) and directing the unreacted isoprenol stream at least partially to oxidative dehydrogenation so as to obtain prenal and/or isoprenal (which may be step c-i)).

In an embodiment, oxidative dehydrogenation is carried out by passing the isoprenol through a plurality of reaction tubes of a shell-and-tube heat exchange reactor comprising:
a shell-side heat exchange passage for circulating a heat transfer medium and a reaction passage comprising the plurality of reaction tubes;
an inlet for introducing the reactant stream to the reaction passage; and
an outlet from the reaction passage for recovering an effluent stream from the reaction tubes;
   wherein the reaction tubes comprise:
   a reactant pre-heating zone adjacent to the inlet, and
   a reaction zone downstream of the reactant pre-heating zone, the reaction zone having a catalytically active wire matrix insert having silver at least on a part of its surface.

The term “reactant pre-heating zone” denotes a section of the reaction tube, i.e., a section inside the reaction tube, where essentially no catalytic oxidative dehydrogenation reaction occurs and where the gaseous stream through the reaction tubes is heat-exchanged via the tube wall with the circulating heat transfer medium. The pre-heating zone upstream of the reaction zone involves net heat flow into the reaction tube and ensures that the reactant stream is sufficiently heated up to a temperature close to or at the reaction temperature when it reaches the reaction zone.

Upon contact with the catalytic surface, the oxidative dehydrogenation reaction immediately starts. Otherwise, in the event when a “cold” reactant stream reaches the catalytic surface such that the reaction onset temperature of the reaction is not reached, coke formation may occur. Less coke formation advantageously leads to a prolonged reactor operation without the necessity of burning off the coke from the catalytic surface.

Preferably, the reactant pre-heating zone is adapted to allow for laminar flow of the reactant inside the reactant pre-heating zone. This means, the reactant pre-heating zone is devoid of any obstacles to the reactant flow that triggers a laminar-to-turbulent flow transition. Hence, the reactant pre-heating zone preferably has an essentially free cross section, i.e., the pre-heating zone is empty.

In the case of an “essentially free cross section”, the reactant pre-heating zone may be empty. Alternatively, the reactant pre-heating zone may accommodate fixtures made of a material having zero or limited catalytic activity, which fixtures have a negligible cross-section in a plane perpendicular to the longitudinal axis of the reaction tube. Said fixtures may be attached to the catalytically active wire matrix which is present in the reaction zone and allow to easily place said wire-matrix insert into or remove the same from the reaction zone. For example, the negligible mounting may be a stainless steel wire or rod.

This setup allows for heating up only the portion of the entire reactant stream that travels near the hot reaction tube wall. Consequently, the portion of the reactant stream flowing in the center of the reaction tube is not heated to the reaction temperature and blind reactions of the unstable starting materials are thus reduced or even avoided. A “blind reaction” is an unselective oxidative reaction that occurs in the absence of the catalyst. Once the reactant stream reaches the reaction zone, the oxidative dehydrogenation reaction is initiated. Due to the exothermic nature of this reaction, energy is released and the remainder of the reactant stream is rapidly heated to the reaction onset temperature, and the reaction proceeds. This fast heat up of the predominant part of the reaction mixture reduces unwanted side-reactions and thus leads to an increased selectivity.

Alternatively, the reactant pre-heating zone may have a wire matrix insert having zero or limited catalytic activity. The wire matrix insert may reduce or eliminate temperature gradients without creating any obstruction to flow that would promote turbulent flow characteristics. A wire matrix insert is considered as having zero catalytic activity (or in other words, as being “inert”) if it does not catalyze the gas-phase partial oxidation reaction in question to a significant degree, and the chemical composition of a stream passing the wire matrix insert does not change significantly.

Similarly, a matrix insert is considered as having limited catalytic activity if its catalytic activity is less than the activity of a reaction zone. In an embodiment, the wire matrix insert having zero or limited catalytic activity is made of an inert material, preferably stainless steel.

Herein, the term “reaction zone” denotes a region of the reaction tube where the catalytic gas-phase partial oxidation reaction occurs. The reaction zone comprises a catalytically active wire matrix insert having at least on a part of its surface a catalytically active precious metal. Due to the more open structure of the wire matrix contained in the reaction zone as compared to a packing of individual elements, a larger proportion of the reaction heat is discharged to the reaction tube wall by radiation and does not have to be dissipated by the reactant stream. Due the unique flow characteristic of the reactant stream through the reaction tube with the wire matrix insert in place, heat transfer via the tube wall is improved. Formation of prominent hotspots can be avoided. This in turn, avoids deposition of organic constituents of the reactant stream on the surface of the active catalyst material with concomitant pressure drop. Overall, less regular maintenance in the form of regeneration and/or replacement of the catalyst is required. The number of annual operating hours can be increased and the existing production capacities can be fully utilized, reducing operation cost and increasing profit.

In contrast to individually present catalyst bodies, the wire matrix inserts can be formed contiguously, or in one piece. Hence, placing the wire matrix inserts in the catalyst containment region of the reaction tubes, and removal therefrom is much facilitated.

The “reaction zone” may be comprised of a single contiguous reaction zone. Alternatively, the reaction zone may comprise an alternating series of regions having catalytically active wire matrix inserts and regions having an essentially free cross section or having wire matrix inserts having zero or limited catalytic activity.

A “wire matrix insert” is understood to be a self-supporting skeletal-like structure made of coiled, bent or crimped metal wire which is adapted to be inserted into a reaction tube of a shell-and-tube reactor. The wire matrix insert has a more voluminous structure than a longitudinal wire.

A fixture such as a stainless steel wire or rod may be attached to the wire matrix insert which allows for easily placing the wire-matrix insert into or removing the same from the reaction zone.

In an embodiment, the catalytically active wire matrix inserts comprise an elongated core having a plurality of wire loops extending from the elongated core, wherein the wire loops are longitudinally arranged and helically shifted, that is, neighboring wire loops have an angular offset. The loops may be formed by helically bending the wire over the length of the wire matrix insert. In view of the ease of manufacture, the elongated core preferably comprises at least two longitudinal core wire members, which are twisted around each other to form core wire windings, and the wire loops are accommodated in the core wire windings.

The wire loops may be formed from one wire, or more than one intertwined wires, preferably 4 intertwined wires.

The wire matrix insert comprised in the reaction zone has silver at least on a part of its surface a catalytically active precious metal. The wire constituting the wire loops may be a massive silver wire, or a wire coated with silver. The core wire may be made of brass alloys, or high-grade steels. The coating layer of silver superimposed on the surface of the core has a thickness of, e.g., 10 μm. In general, however, a massive silver wire has better service life and is preferred. If the wire loops are formed from more than one intertwined wires, at least one of the intertwined wires is made of a massive silver wire, or a wire coated with silver while the other intertwined wires can be made of an inert material.

A silver wire which is of the same composition throughout its cross section and comprises at least 92.5 wt.-% Ag can suitably be used. The silver wire is helically bent to form wire loops, and combined with at least two longitudinal core wire members, which are twisted around each other to form core wire windings, and the wire loops are accommodated in the core wire windings. The longitudinal core wire members can also be silver wire or inert metal wire.

In a preferred embodiment, the catalytically active wire matrix inserts comprise an elongated core having a plurality of wire loops extending from the elongated core, wherein the wire loops are longitudinally arranged and helically shifted, and the wire loops comprise a massive silver wire.

Generally, the catalytically active wire matrix inserts may have a cylindrical enveloping surface with a diameter matching with the inner diameter of the reaction tubes. This may include a situation where the diameter of the cylindrical enveloping surface of the undeployed wire matrix insert is slightly larger than the inner diameter of the reaction tubes. Due to the springy or elastic nature of the wire matrix insert, it can be inserted into the reaction tubes with a slight counter pressure such that the wire loops fit tightly against the inner walls of the reaction tube.

Suitable structures of wire matrix inserts are known as such, see, e.g., GB 2 097910 Some inserts of this type are disclosed in GB patent 1 570 530. Other inserts, as well as processes for their production are disclosed in GB 2 097 910 A. Matrix inserts are commercially available from the company Cal Gavin Ltd., England, and sold under the trade name HiTRAN®.

Further details of oxidative dehydrogenation carried out by passing the isoprenol through a plurality of reaction tubes of a shell-and-tube heat exchange reactor as described above may be found in WO 2023/241952 A1, which herewith is incorporated by reference in its entirety.

When isoprenol is subjected to oxidative dehydrogenation, it may be favorable to maintain in the reactant stream a weight ratio of aldehydes, preferably prenal and/or formaldehyde to isoprenol of less than 0.04, preferably less than 0.03, in particular less than 0.02, or less than 0.01. In another embodiment, the weight ratio of aldehydes, preferably prenal and/or formaldehyde to isoprenol is maintained at less than 0.002, or less than 0.001 and optionally at least 100 ppm.

The weight ratio of aldehydes, preferably prenal and/or formaldehyde to isoprenol in the reactant stream may be maintained at a certain level or less. Reducing the weight ratio of aldehydes, preferably prenal and/or formaldehyde to isoprenol in the reactant stream beyond a certain point, however, reaches a point of rapidly diminishing return. Aldehydes, preferably prenal and/or formaldehyde removal involves additional equipment and operating costs. An economic balance must be taken between the improvement due to reducing the ratio and the cost of maintaining such a ratio. Hence, the weight ratio of aldehydes, preferably prenal and/or formaldehyde to isoprenol is preferably not lower than 0.0005. In an alternative embodiment the weight ratio is not lower than 0.005.

It has been found that reactor clogging and pressure drop increase are significantly affected by the presence of aldehydes, preferably prenal and/or formaldehyde in the reactant stream. Catalyst-fouling reactions of condensation and polymerization are believed to be the principal reactions involved in carbon or coke formation on the catalyst. It is thought that this carbon formation involves thermal condensation of aldehydes, preferably prenal and/or formaldehyde or of aldehydes, preferably prenal and/or formaldehyde with the olefinic hydrocarbons isoprenol and (iso)prenal. In the presence of the catalyst, the primary condensation products tend to undergo dehydrogenation and polymerization type reactions and to settle on the catalyst and undergo further dehydrogenation and decomposition until carbonaceous deposits are formed.

The presence of formaldehyde in the reactant stream is due to two main sources. Formaldehyde may be contained in the fresh feed stream sent to the reactor, that is as an impurity originating from the isoprenol manufacture step. All the formaldehyde that cannot be separated in the purification step following the isoprenol synthesis may end up in the reactant stream.

In addition, formaldehyde is also generated in situ. Part of the isoprenol splits back to isobutene and formaldehyde. Since most continuous industrial processes operate at single-pass conversion levels of 50 to 60% and with recycling of the unconverted isoprenol, formaldehyde may be present in the recycling stream of unconverted isoprenol, if no steps to purify the stream containing unreacted isoprenol are taken. The recycle stream of unconverted isoprenol has now been found to constitute the biggest source of formaldehyde contamination in the reactant stream. The process is generally carried out at partial conversions, for example at conversions of 30 to 70%, preferably 50 to 60%. An unreacted isoprenol stream is separated from the product stream. The unreacted isoprenol stream is recycled, that is, combined with a fresh feed stream comprising isoprenol (a crude isoprenol stream) to provide the reactant stream. The unreacted isoprenol stream comprises isoprenol as a main constituent, but may also comprise prenal, isoprenal, isoamyl alcohol, isovaleraldehyde, isovaleric acid, prenol, formaldehyde. It can also contain traces of other C₃ and C₂ aldehydes and acids.

Reducing the weight ratio of aldehydes, preferably prenal and/or formaldehyde to isoprenol in the reactant stream can be accomplished in several different ways. In an embodiment, aldehydes, preferably prenal and/or formaldehyde, are removed from the unreacted isoprenol stream prior to combining the unreacted isoprenol stream with the crude isoprenol stream.

In an embodiment, the unreacted isoprenol stream is combined with the crude isoprenol stream and aldehydes, preferably prenal and/or formaldehyde is removed from the combined stream.

Alternatively, it is feasible to mix the unreacted isoprenol stream with an amount of a sufficiently purified fresh feed stream so as to give in the combined stream a desired weight ratio of formaldehyde to isoprenol.

Aldehydes, preferably prenal and/or formaldehyde, may be removed from isoprenol streams by a conventional separating method such as distillation, selective adsorption and or selective reaction, in particular by the purification process involving the pressure-swing distillation as described above.

### Pre-Treating (Iso)Prenol by Nitrogen Removal Prior to Oxidative Deyhdrogenation or Oxidation

Prior to contacting with the at least one oxidative dehydrogenation catalyst or with the at least one oxidation catalyst, respectively, the (iso)prenol may advantageously be treated to remove organically bound nitrogen from the (iso)prenol by contacting the (iso)prenol with a weakly acidic solid adsorbent. In other words, the (iso)prenol may be depleted of organically bound nitrogen by this process.

The term “organically bound nitrogen” is intended to denote any compound containing at least one nitrogen atom directly bound to one or more carbon atoms. For example, such compounds containing at least one nitrogen atom may be selected from amines, such as ethylamine, trimethylamine, aniline, pyridine or piperidine. An amine particularly significant in practice is hexamethylenetetramine (urotropin). (Iso)prenol may comprise about 5 to 30 ppm of organically bound nitrogen.

The weakly acidic solid adsorbents have been found to be capable of adsorbing organically bound nitrogen in the presence of abundant (iso)prenol while not interfering with the reactive carbon-carbon double bond.

The weakly acidic adsorbent may include an adsorbent material having sufficient acidity to adsorb the organically bound nitrogen from the (iso)prenol. In an embodiment, the solid adsorbent is a crosslinked resin having phosphonic functional groups. Preferably, the resin polymer is a vinyl aromatic copolymer, preferably crosslinked polystyrene and more preferably a polystyrene divinylbenzene copolymer. Other polymers having a phosphonic functional group may also be used. Preferably, the crosslinked resin having phosphonic functional groups is of the macroporous type. A preferred solid adsorbent is Purolite S956.

The resin is typically used in bead form and loaded into a column. The (iso)prenol is passed through the column, contacting the resin beads. During contact, the organically bound nitrogen in the (iso)prenol reacts with the functional group and an exchange occurs where a proton is transferred to the nitrogen and an ionic bond is formed to the anionic site of the resin. Contact is maintained until a threshold level is reached i.e., the breakthrough concentration. At this breakthrough point, the process reaches an equilibrium where additional organically bound nitrogen cannot be removed effectively. The flow is held/stopped and the column is backwashed with water, preferably deionized or softened water. By flowing in reverse, the resin is fluidized and solids captured by the beads are loosened and removed.

In another embodiment, the solid adsorbent is a silica-alumina hydrate. Numerous silica-alumina catalyst compositions and processes for their preparation are described in the patent literature, see, e.g., U.S. Pat. No. 4,499,197.

Preferably, the alumina content of the silica-alumina hydrate is from about 10 to about 90 wt.-% of Al₂O₃. The preferred range of alumina content is from about 30 to about 70 wt.-% of Al₂O₃.

The introduction of silicon dioxide into aluminum oxide leads to the introduction of acidic centers. The number of acidic centers can be controlled by the amount of introduced silicon dioxide. The number of acidic centers increases with the amount of introduced silicon dioxide up to a maximum number of acidic centers, and decreases again with a further increasing amount of silicon dioxide after having reached the maximum number of acidic centers.

Examples of commercially available silica-alumina hydrates are Siral® available from Sasol Germany Gmbh, Hamburg, Germany. Siral® is based on orthorhombic aluminum oxide hydroxide (boehmite; AlOOH) and doped with SiO₂. Various Siral® grades having different ratios of Al₂O₃ to SiO₂ are available: Siral 1 (Al₂O₃/SiO₂=99/1), Siral 5 (Al₂O₃/SiO₂=95/5), Siral 10 (Al₂O₃/SiO₂=90/10), Siral 20 (Al₂O₃/SiO_{2=80/20}), Siral 28M (Al₂O₃/SiO₂=72/28), Siral 30 (Al₂O₃/SiO₂=70/30), Siral 40 (Al₂O₃/SiO₂=60/40). Siral 40 is especially preferred.

In an embodiment, the (iso)prenol is passed over a bed of the weakly acidic solid adsorbent. Suitably, said step of “passing over a bed” denotes that a layer (“bed”) of the weakly acidic solid adsorbent is provided in a customary reaction vessel known to the skilled person which may preferably be equipped with a stirring device, e.g., in a stirred-tank reactor. The (iso)prenol is then introduced into the reaction vessel and guided through the same in a manner that it gets into contact with the weakly acidic solid adsorbent.

Alternatively, the weakly acidic solid adsorbent may be provided in a reaction tube, e.g., of a tubular reactor and the (iso)prenol then continuously flows through said reaction tube(s) while getting into contact with the weakly acidic solid adsorbent.

In an alternative embodiment, the (iso)prenol comprises, after contacting the alcohol stream with a weakly acidic solid adsorbent, less than 2 ppm of organically bound nitrogen. Herein, “ppm” denotes wt.-ppm of compounds incorporating organically bound nitrogen, relative to the total weight of the (iso)prenol.

For example, the content of organically bound nitrogen in the (iso)prenol may be determined by Kjeldahl analysis. Alternatively, an oxidative combustion method with a chemiluminescence detector according to DIN 51444 may be used.

In an embodiment, the process for preparing prenal is a continuous process. In an embodiment, all steps for preparing prenal are conducted continuously, in particular are conducted as consecutive continuous steps. This may for instance include steps for preparing isoprenol (e.g., from formaldehyde and isobutene), oxidation to prenal and/or isoprenal, optionally converting isoprenal to prenal, and optional one or more purification steps.

### Further Conversion to Citral

The prenal produced in a step of subjecting diprenyl acetal to cleaving conditions to obtain citral (which may be step c)) may is useful in the preparation of citral. Citral is a mixture of the isomeric compounds neral and geranial.

3,7-dimethyl-octa-2,6-dienal (citral) can be prepared by obtaining prenal by a process as described above, further comprising the steps of condensing the prenal with prenol to obtain diprenyl acetal of prenal; and subjecting the diprenyl acetal of prenal to cleaving conditions to obtain citral via prenyl (3-methyl-butadienyl) ether and 2,4,4-trimethyl-3-formyl-1,5-hexadiene.

In particular, 3,7-dimethyl-octa-2,6-dienal (citral) can be prepared by a process comprising the steps of:
condensing the prenal with prenol in the presence of at least one catalyst in a reaction column while withdrawing an acetal fraction comprising the diprenyl acetal of prenal from the reaction column;
subjecting the acetal fraction in a cleaving column to cleaving conditions in the presence of at least one catalyst while withdrawing from the cleaving column a cleaving fraction containing at least one of prenyl (3-methyl-butadienyl) ether and 2,4,4-trimethyl-3-formyl-1,5-hexadiene, and optionally containing citral; and
reacting the cleaving fraction in a plug-flow type reactor to obtain citral.

The overall reaction sequence is illustrated by the reaction scheme below.

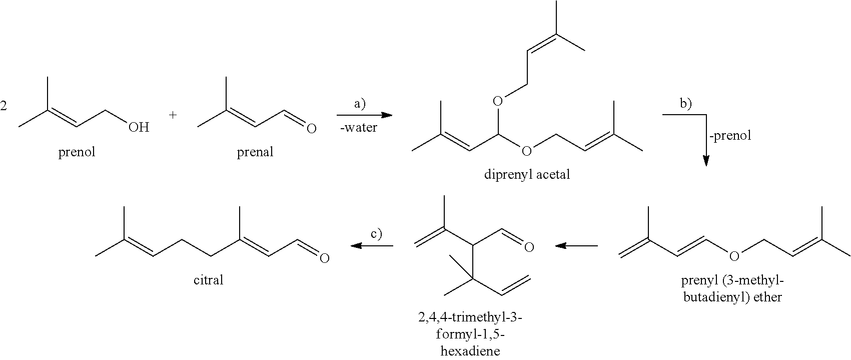

The unsaturated acetal 3-methyl-2-butenal-diprenyl acetal (herein referred to as “diprenyl acetal of prenal” or “diprenyl acetal”) is formed from prenol and prenal using at least one catalyst. For this purpose, prenal may be reacted together with prenol in the presence of catalytic amounts of at least one acid and with separation of the water formed during the reaction in a reaction column.

It has been found that when the conversion rate of diprenyl acetal of prenal is driven to full conversion, the concentration of by-products increases sharply. Accordingly, it is preferred that the conversion rate of diprenyl acetal of prenal is maintained at above 90% and below 100%. Preferably, the conversion rate of diprenyl acetal of prenal (which may be conducted as in step b)) is maintained equal to or below 99.5%, preferably equal to or below 99%, such as equal to or below 98%, or equal to or below 97.5%, or equal to or below 97%. Preferably, the conversion rate of diprenyl acetal of prenal is maintained above 91%, such as above 92%, or above or 93%, or above 94%, or above 95%. In suitable embodiments, the conversion rate of diprenyl acetal of prenal in is above 94% and equal to or below 99%, such as above 95% and equal to or below 98%. Lower conversion rates will render the process economically unprofitable or will otherwise necessitate recovery and recycling of unreacted diprenyl acetal. Complete conversion is however undesirable as it results in a drop of yield of citral building blocks and increasing by-products-formation. The conversion rate is governed by various parameters including cleaving temperature, nature and concentration of the catalyst(s) and residence time in the cleaving column.

The resulting 3-methyl-2-butenal diprenyl acetal (diprenyl acetal) is cleaved in the presence of at least one catalyst in a cleaving column with elimination of 3-methyl-2-buten-1-ol (prenol) to give prenyl (3-methylbutadienyl) Claisen rearrangement of the obtained prenyl (3-methylbutadienyl) ether yields 2,4,4-trimethyl-3-formyl-1,5-hexadiene which subsequently undergoes Cope rearrangement yielding 3,7-dimethyl-2,6-octadienal (citral).

Cleaving is carried out in the presence of at least one catalyst, preferably an acid catalyst. The catalyst can be a single catalytic species or a combination of two or more different catalytic species. Suitable acid catalysts are selected from non-volatile protic acids such as sulfuric acid, p-toluenesulfonic acid and phosphoric acid. In an embodiment, the catalyst comprises phosphoric acid. In a preferred embodiment, the concentration of the phosphoric acid in the bottoms of the cleaving column is maintained above 100 ppm and below 1500 ppm, preferably above 200 ppm and below 1000 ppm. Higher concentrations of (acid) catalyst may result in reduced yields of citral building blocks.

Condensation of prenol with prenal is carried out in the presence of at least one catalyst, preferably an acid. The catalyst can be a single catalytic species or a combination of two or more different catalytic species. In an embodiment, the catalyst in is nitric acid. Preferably, the concentration of the nitric acid is below 500 ppm, more preferably in the range of from 100 to 300 ppm, relative to the total amount of the starting materials prenol and prenal. Lower amounts of (acid) catalyst may result in a low conversion in the reaction column. Higher amounts of (acid) catalyst may disadvantageously result in increased formation of by-products and in decreased selectivity.

Preferably, the acetal fraction is continuously subjected to cleaving conditions in a cleaving column. “Cleaving conditions” denotes reaction conditions selected such that the diprenyl acetal contained in the acetal fraction is cleaved to prenyl (3-methylbutadienyl) ether which may subsequently rearrange to 2,4,4-trimethyl-3-formyl-1,5-hexadiene and citral.

The acetal fraction comprises diprenyl acetal as a main constituent. The acetal fraction does not necessarily need to consist of pure diprenyl acetal, but may also comprise prenol, prenal and citral building blocks.

Cleaving is carried out in the presence of at least one catalyst, preferably at least one acid catalyst. Suitable acid catalysts are selected from non-volatile protic acids such as sulfuric acid, p-toluenesulfonic acid and phosphoric acid.

Suitably, the continuous cleaving in the cleaving column may be carried out in the lower part or the sump of the distillation column acting as cleaving column. Preferably, the acetal fraction and/or the catalyst(s) are introduced into the lower part of the distillation column, into the sump of the distillation column or into the evaporator of the distillation column.

If desired, a high-boiling inert compound can be introduced into the sump of the cleaving column in order to ensure a minimum filling level of the sump and the evaporator. Suitable high-boiling inert compounds are selected from liquid compounds which are inert under the reaction conditions and have a higher boiling point than citral and diprenyl acetal. For example, the high-boiling inert compounds may be selected from hydrocarbons such as tetradecane, pentadecane, hexadecane, octadecane, eicosane; or ethers such as diethylene glycol dibutyl ether; white oils; kerosene oils; or mixtures thereof.

Suitably, the distillation conditions are selected such that the diprenyl acetal is predominantly retained in the lower part or the sump of the distillation column. During the cleaving reaction, a cleaving fraction is continuously withdrawn from the cleaving column, the cleaving fraction containing at least one of prenyl (3-methyl-butadienyl) ether and 2,4,4-trimethyl-3-formyl-1,5-hexadiene, and optionally containing citral. For the ease of reference, prenyl (3-methyl-butadienyl) ether, 2,4,4-trimethyl-3-formyl-1,5-hexadiene and citral are collectively referred to as “citral building blocks”. This is because the former are intermediates on the reaction route to citral and can be converted into citral in the subsequent passage through the plug-flow type reactor.

Additionally, the prenol formed during the cleaving reaction may be continuously removed from the reaction mixture, generally at the top of the cleaving column.

The cleaving fraction together with the formed prenol may be withdrawn at the top of the distillation column.

Alternatively and preferably, it is also possible to withdraw the cleaving fraction in liquid or vaporous form at a side draw of the distillation column.

The cleaving fraction may be reacted in a plug-flow type reactor to obtain citral. To this end, the cleaving fraction is guided through the plug-flow type reactor at a suitable temperature for carrying out the rearrangement reaction(s) yielding citral. By employing a combination of a highly back-mixed cleaving column and a plug-flow reactor, it is possible to increase the selectivity and the yield of the cleaving reaction. All of the catalyst(s) required for the cleaving reaction is/are preferably introduced into the cleaving column and preferably, no catalyst is introduced into the plug-flow reactor.

In an embodiment, prenol eliminated in the cleaving reaction is recycled to the condensation reaction. This allows for improved yields to be achieved in the process of the invention.

In particular, the inventive process may comprise recycling prenol obtained in a step of condensing prenol with prenal to obtain diprenyl acetal of prenal (which may be step d)) to a step of subjecting diprenyl acetal of prenal to cleaving conditions to obtain citral (which may be step e)), in particular comprising recycling prenol obtained in step e) to step d);
wherein the concentration of 2,4,4-trimethyl-3-formyl-1,5-hexadiene of the prenol recycled from step e) into step d) is controlled such that the concentration of 2,4,4-trimethyl-3-formyl-1,5-hexadiene in step d) is below 1 wt.-%, relative to the total weight of prenol and prenal; and
wherein the concentration of citral of the prenol recycled (in particular from step e) into step d)) is controlled such that the concentration of citral (in particular in step d)) is below 1 wt.-%, relative to the total weight of prenol and prenal.

The present invention refers to a process for the preparation of 3,7-dimethyl-octa-2,6-dienal (citral) comprising a step subjecting diprenyl acetal of prenal to cleaving conditions to obtain citral via prenyl (3-methyl-butadienyl) ether and 2,4,4-trimethyl-3-formyl-1,5-hexadiene, wherein at least one of the prenal and/or the prenol is obtained from a process of the present invention. In a preferred embodiment, in this process for preparing citral, the diprenyl acetal of prenal is obtained from a process of the present invention.

In an embodiment, the process for preparing citral is a continuous process. In an embodiment, steps for preparing citral are conducted continuously, in particular are conducted as consecutive continuous steps. This may for instance include steps for preparing prenal (which may be steps a) and c)) and/or prenol (which may be steps a) and b)) as laid out above, condensing prenol with prenal to obtain diprenyl acetal of prenal (which may be step d)), rearrangement of citral (which may be step e)), and optional one or more purification steps.

### Further Aspect of the Invention: Process for Preparing Prenal

It will be understood that the definitions and embodiments defined for a process for the preparation of citral and/or prenol mutatis mutandis apply to a process for preparing prenal.

In embodiments of the present invention, the prenol is obtained from isomerizing isoprenol to obtain prenol by bringing a reactant stream comprising isoprenol into contact with at least one heterogeneous isomerization catalyst, preferably in the presence of hydrogen.

In embodiments of the present invention, the isoprenol is obtained from reacting at least one formaldehyde source and isobutylene so as to obtain the isoprenol.

In embodiments of the present invention, the step of isomerizing isoprenol is characterized by maintaining in the reactant stream a concentration of aldehydes of less than 0.5 wt.-%, preferably less than 0.4 wt.-%, in particular less than 0.3 wt.-%, or less than 0.25 wt.-%, based on the total weight of the reactant stream, and, optionally, the concentration of aldehydes in the reactant stream is not lower than 10 ppm, preferably not lower than 25 ppm, in particular not lower than 50 ppm, or not lower than 100 ppm, based on the total weight of the reactant stream.

### Further Aspect of the Invention: Process for Preparing Diprenyl Acetal of Prenal

It will be understood that the definitions and embodiments defined for a process for the preparation of citral, prenol and/or prenal mutatis mutandis apply to a process for preparing diprenyl acetal of prenal.

In embodiments of the present invention, the invention provides a process for the preparation of diprenyl acetal of prenal comprising the step of condensing prenal with prenol, wherein at least one of prenal and/or prenol is obtainable (or obtained) according to a process of the present invention.

In embodiments of the present invention, the process for preparation of prenal comprises:
a) reacting at least one formaldehyde source and isobutylene to obtain isoprenol;
b) isomerizing isoprenol obtained in step a) to obtain prenol by bringing a reactant stream comprising isoprenol into contact with at least one heterogeneous isomerization catalyst, preferably in the presence of hydrogen;
c) providing prenal according to a process of the present invention; and
d) condensing prenol obtained in step b) with prenal obtained in step c) to obtain diprenyl acetal of prenal.

### Further Aspect of the Invention: Product Stream Containing Prenal

It will be understood that the definitions and embodiments defined for the processes of the present invention mutatis mutandis apply a product stream containing prenal.

Due to the particularly high selectivity in the step of oxidative dehydrogenation of isoprenol to prenal and/or isoprenal in a process of the present invention, it will be understood that the obtainable (obtained) product steam containing prenal contains particularly pure prenal in the product stream and less undesired side products. The content ranges and side products in the product stream may be altered due to limiting the content of aldehydes in the reactant stream.

### Further Aspect of the Invention: Product Stream Containing Prenol

It will be understood that the definitions and embodiments defined for the processes of the present invention mutatis mutandis apply a product stream containing prenol.

Due to the particularly high selectivity in the step of isomerizing isoprenol to prenol in a process of the present invention, it will be understood that the obtainable (obtained) product steam containing prenol contains particularly pure prenol in the product stream and less undesired side products. The content ranges and side products in the product stream may be altered due to limiting the content of aldehydes in the reactant stream.

### Further Aspect of the Invention: Product Stream Containing Diprenyl Acetal of Prenal

It will be understood that the definitions and embodiments defined for the processes of the present invention mutatis mutandis apply a product stream containing diprenyl acetal of prenal.

Due to the particularly high selectivity in the step of oxidative dehydrogenation of isoprenol to prenal and/or isoprenal in a process of the present invention, and/or the particularly high selectivity in the step of isomerizing isoprenol to prenol in a process of the present invention, it will be understood that the obtainable (obtained) product steams containing prenal and/or prenol contain particularly pure prenal and/or prenol in the product stream. It will be understood that this may evidently lead to particularly pure diprenyl acetal of prenal in the product stream and less undesired side products. The content ranges and side products in the product stream may be altered due to limiting the content of aldehydes in the one or more reactant streams.

### Further Aspect of the Invention: Product Stream Containing Citral

It will be understood that the definitions and embodiments defined for the processes of the present invention mutatis mutandis apply a product stream containing citral.

Due to the particularly high selectivity in the step of oxidative dehydrogenation of isoprenol to prenal and/or isoprenal in a process of the present invention, and/or the particularly high selectivity in the step of isomerizing isoprenol to prenol in a process of the present invention, it will be understood that the obtainable (obtained) product steams containing prenal and/or prenol contain particularly pure prenal and/or prenol in the product stream. It will be understood that this may evidently lead to particularly pure citral in the product stream and less undesired side products. The content ranges and side products in the product stream may be altered due to limiting the content of aldehydes in the one or more reactant streams.

In an embodiment, the process for preparing prenal and/or prenol is a continuous process. In an embodiment, all steps for preparing citral are conducted continuously, in particular are conducted as consecutive continuous steps.

Citral occurs as (2Z)- and (2E)-isomers: the (2Z))-Isomer, neral, as depicted in formula (Vb-1) and the (2E)-Isomer, geranial, as depicted in formula (Va-1).

The term citral obtainable (or obtained) according to the method of the present invention may be any mixture of the two isomers, preferably a mixture having a mass ratio of neral:geranial of between 40. 60 to 60:40, in particular between 45. 55 to 55:45, between 48. 52 to 52:48, between 49. 51 to 51:49, or (approximately) 50:50.

As known in the art, neral and geranial may be optionally separated from one another. For instance, neral and geranial may be separated by distillation. This allows adjusting the mass ratio of neral:geranial to a desired degree.

Citral obtainable (or obtained) according to the invention may comprise geranial of the formula (Va-1) and/or of neral of the formula (Vb-1).

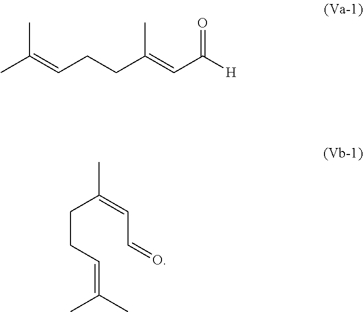

A further aspect of the present invention relates to geranial of the formula (Va-1), neral of the formula (Vb-1) or a mixture thereof obtainable (or obtained) from a method of the present invention.

It will be understood that the definitions and preferred embodiments as laid out in the context of preparing (iso)prenal and the further steps directed to citral above mutatis mutandis apply to the preparation and product characteristics of geranial of the formula (Va-1), neral of the formula (Vb-1) or a mixture thereof. It will be understood that the geranial of the formula (Va-1), neral of the formula (Vb-1) and a mixture thereof have certain characteristics.

### Further Conversion to Menthol or Linalool

The obtainable (obtained) citral is a useful intermediate for, e.g., menthol or linalool. It will be understood that also such products may bear particularly beneficial characteristics such as purity when the citral is obtained from a process of the present invention.

Menthol (p-menthal-3-ol) is a naturally occurring active ingredient that is widely used in pharmaceuticals, cosmetics and the food industry. Menthol has a cooling effect when it comes into contact with mucous membranes, especially the oral mucosa. In natural sources, for example peppermint oil, menthol occurs in the form of four diastereomeric enantiomer pairs. The following formula depicts the main component, (−)-menthol or L-menthol, which has desired taste and other sensory properties.

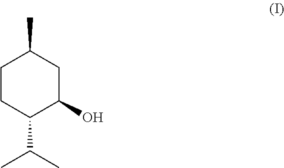

IUPAC name: 1R,2S,5R2-isopropyl-5-methylcyclohexanol

Menthol may be prepared from citral via a process comprising the steps of
catalytic hydrogenation of citral to obtain citronellal;
cyclization of citronellal to obtain isopulegol in the presence of at least one acidic catalyst; and
catalytic hydrogenation of isopulegol to obtain menthol.

The overall reaction sequence is illustrated by the reaction scheme below.

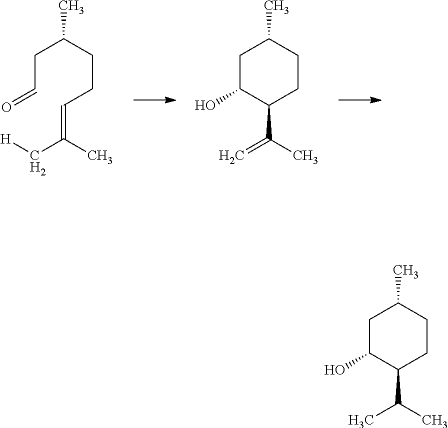

The hydrogenation of citral to obtain citronellal may be achieved by hydrogenation in the presence of a rhodium-phosphine catalyst.

A further aspect of the invention is directed to a process for preparation of menthol comprising the steps of:
catalytic hydrogenation of citral obtainable (or obtained) according to the invention, preferably according to any of the claims 8 to 19, 31 and 32, to obtain citronellal;
cyclization of citronellal prepared in this way to obtain isopulegol in the presence of at least one acidic catalyst; and
catalytic hydrogenation of isopulegol prepared in this way to obtain menthol.

A further aspect of the present invention is directed to a process for the preparation of optically active menthol using citral obtained by the process according to the invention.

A further aspect of the invention is directed to a process for the preparation of optically active menthol, preferably L-menthol, comprising the steps of 0) optionally separating the citral obtainable (or obtained) according to the invention, preferably according to any of the claims 8 to 19, 31 and 32, into geranial of the formula (Va-1) and neral of the formula (Vb-1))
i) preparation of optically active citronellal by asymmetric hydrogenation of citral obtainable (or obtained) according to the invention, preferably according to any of the claims 8 to 19, 31 and 32, geranial of the formula (Va-1) or of neral of the formula (Vb-1) by the process according to the invention,
ii) cyclization of the optically active citronellal prepared in this way to give optically active isopulegol in the presence of a suitable acid, preferably a Lewis acid, and
iii) hydrogenation of the optically active isopulegol prepared in this way to give optically active menthol.

A further aspect of the present invention relates to menthol, which may be optionally optically active menthol, preferably L-menthol, obtainable (or obtained) from a method of the present invention.

It will be understood that the definitions and preferred embodiments as laid out in the context of preparing (iso)prenal and/or prenol, citral and further steps and obtainable (or obtained) products above mutatis mutandis apply to the preparation and product characteristics of menthol. It will be understood that the obtainable (or obtained) menthol has certain characteristics.

The cyclization of citronellal to isopulegol may be achieved by cyclization in the presence of at least one Lewis-acidic aluminum-containing catalyst, such as a bis(diarylphenoxy)aluminum compound, which may be used in the presence of an auxiliary, such as a carboxylic anhydride. The isopulegol may be recovered from the catalyst-containing reaction product by distillative separation to give an isopulegol-enriched top product and an isopulegol-depleted bottom product. From the bottom product, the at least one catalyst may be regenerated. The isopulegol obtainable in this way by the cyclization of citronellal can be further purified by suitable separating and/or purification methods, in particular by crystallization, and be at least largely freed from undesired impurities or by-products.

The hydrogenation of isopulegol may be achieved by hydrogenation in the presence of at least one heterogeneous nickel-containing catalyst, preferably at least one heterogeneous nickel- and copper-containing catalyst.

Further details regarding the reaction sequence from citral to menthol may be found in US 2013/46118 A1, which is incorporated by reference herein.

Isopulegol (5-methyl-2-(1-methylethenyl)-cyclohexanol) has three asymmetric carbon atoms and therefore four stereoisomers, each occurring as a pair of enantiomers. (1R,3R,4S)-(−)isopulegol is also known as L-isopulegol.

A further aspect of the invention is directed to a process for the preparation of isopulegol, preferably optically active isopulegol, preferably L-isopulegol, comprising the steps of
o) optionally separating the citral obtainable (or obtained) according to the invention, preferably according to any of the claims 8 to 19, 31 and 32, into geranial of the formula (Va-1) and neral of the formula (Vb-1)),
i) preparation of optically active citronellal by asymmetric hydrogenation of citral obtainable (or obtained) according to the invention, preferably according to any of the claims 8 to 19, 31 and 32, geranial of the formula (Va-1) or of neral of the formula (Vb-1) by the process according to the invention,
ii) cyclization of the optically active citronellal prepared in this way to give optically active isopulegol in the presence of a suitable acid, preferably a Lewis acid.

A further aspect of the present invention relates to isopulegol, which may be optionally optically active isopulegol, preferably L-isopulegol, obtainable (or obtained) from a method of the present invention.

It will be understood that the definitions and preferred embodiments as laid out in the context of preparing (iso)prenal and/or prenol, citral and further steps and obtainable (or obtained) products above mutatis mutandis apply to the preparation and product characteristics of isopulegol. It will be understood that the obtainable (or obtained) isopulegol has certain characteristics.

In one aspect, the invention thus relates to an improved process for the preparation of menthol by producing citral using the above processes and then producing menthol from the citral. Menthol may be prepared as described herein or by other methods known in the art.

Linalool may be prepared from citral via a process comprising catalytic hydrogenation of citral to obtain nerol and/or geraniol, and isomerization thereof.

The hydrogenation of citral to obtain nerol and/or geraniol may be achieved by hydrogenation in the presence of at least one supported ruthenium, rhodium, osmium, iridium or platinum catalyst, preferably at least one ruthenium catalyst supported on carbon black.

The isomerization of nerol and/or geraniol to obtain linalool may be achieved by isomerization in the presence of at least one tungsten catalyst, in particular a dioxotungsten (VI) complex. Further details regarding the isomerization of nerol and/or geraniol may be found in U.S. Pat. No. 7,126,033 B2.

In one aspect, the invention thus relates to an improved process for the preparation of linalool by producing citral using the above processes and then producing linalool from the citral. Linalool may be prepared as described herein or by other methods known in the art.

Isopulegol (5-methyl-2-(1-methylethenyl)-cyclohexanol) has three asymmetric carbon atoms and therefore four stereoisomers, each occurring as a pair of enantiomers. (1R,3R,4S)-(−) isopulegol is also known as L-isopulegol.

A further aspect of the invention is directed to a process for the preparation of isopulegol, preferably optically active isopulegol, preferably L-isopulegol, comprising the steps of
o) optionally separating the citral obtainable (or obtained) according to the invention, preferably according to any of the claims 8 to 19, 31 and 32, into geranial of the formula (Va-1) and neral of the formula (Vb-1)),
i) preparation of optically active citronellal by asymmetric hydrogenation of citral obtainable (or obtained) according to the invention, preferably according to any of the claims 8 to 19, 31 and 32, geranial of the formula (Va-1) or of neral of the formula (Vb-1) by the process according to the invention,
ii) cyclization of the optically active citronellal prepared in this way to give optically active isopulegol in the presence of a suitable acid, preferably a Lewis acid.

A further aspect of the present invention relates to isopulegol, which may be optionally optically active isopulegol, preferably L-isopulegol, obtainable (or obtained) from a method of the present invention.

It will be understood that the definitions and preferred embodiments as laid out in the context of preparing (iso)prenal and/or prenol, citral and further steps and obtainable (or obtained) products above mutatis mutandis apply to the preparation and product characteristics of isopulegol. It will be understood that the obtainable (or obtained) isopulegol has certain characteristics.

A further aspect of the present invention relates to linalool obtainable (or obtained) from a method of the present invention.

It will be understood that the definitions and preferred embodiments as laid out in the context of preparing (iso)prenal and/or prenol, citral and further steps and obtainable (or obtained) products above mutatis mutandis apply to the preparation and product characteristics of linalool. It will be understood that the obtainable (or obtained) linalool has certain characteristics.

A further aspect of the invention is directed to a process for the preparation of vitamin A acetate comprising the steps of
converting Citral (VII) obtainable (or obtained) according to the invention, preferably according to any of the claims 8 to 19, 31 and 32, into pseudoionone (VIII),
reacting pseudoionone (VIII) to obtain β-ionone (IX),
transforming β-ionone (IX) into β-vinylionol of formula (X),
phosphorylation of β-vinylionol of formula (X) to yield the C₁₅-salt of formula (XI), and
reacting the C₁₅-salt of formula (XI) with the C₅-acetate of formula (XII) to yield vitamin A acetate of formula (XIII).

A further aspect of the present invention relates to vitamin A obtainable (or obtained) from a method of the present invention.

It will be understood that the definitions and preferred embodiments as laid out in the context of preparing (iso)prenal and/or prenol, citral and further steps and obtainable (or obtained) products above mutatis mutandis apply to the preparation and product characteristics of vitamin A. It will be understood that the obtainable (or obtained) vitamin A has certain characteristics.

In general, the present invention further refers to the process for the preparation of 3,7-dimethyl-octa-2,6-dienal (citral) comprising the steps of:
a) reacting a formaldehyde source and isobutylene to obtain isoprenol;
b) isomerizing isoprenol to obtain prenol;
c) converting isoprenol to prenal, involving isomerization and an oxidative dehydrogenation in any order;
d) condensing prenol with prenal to obtain diprenyl acetal of prenal;
e) subjecting diprenyl acetal of prenal to cleaving conditions to obtain citral via prenyl (3-methyl-butadienyl) ether and 2,4,4-trimethyl-3-formyl-1,5-hexadiene.

The invention is further illustrated by the following embodiments.
1. A process for the preparation of 3,7-dimethyl-octa-2,6-dienal (citral) comprising the steps of:
   a) reacting at least one formaldehyde source and isobutylene to obtain isoprenol;
   b) isomerizing isoprenol obtained in step a) to obtain prenol by bringing a reactant stream comprising isoprenol into contact with at least one heterogeneous isomerization catalyst, preferably in the presence of hydrogen;
   c) providing prenal by at least one of c-i) and c-ii):
      c-i) subjecting isoprenol obtained in step a) to oxidative dehydrogenation so as to obtain prenal and/or isoprenal by bringing a reactant stream comprising isoprenol into contact with at least one heterogeneous oxidative dehydrogenation catalyst, in the presence of molecular oxygen, and optionally isomerizing at least part of the isoprenal to prenal;
      c-ii) oxidizing prenol obtained in step b) so as to obtain prenal by bringing a reactant stream comprising prenol into contact with at least one oxidant and at least one oxidation catalyst, preferably in the presence of a liquid phase;
   d) condensing prenol obtained in step b) with prenal obtained in step c) to obtain diprenyl acetal of prenal; and
   e) subjecting diprenyl acetal of prenal obtained in step d) to cleaving conditions to obtain citral via prenyl (3-methyl-butadienyl) ether and 2,4,4-trimethyl-3-formyl-1,5-hexadiene;
   wherein at least the step c-i) is characterized by maintaining in the reactant stream a weight ratio of aldehydes, preferably prenal and/or formaldehyde to isoprenol of less than 0.04,
   wherein step b) is characterized by maintaining in the reactant stream a concentration of aldehydes of less than 0.5 wt.-%, preferably less than 0.4 wt.-%, in particular less than 0.3 wt.-%, or less than 0.25 wt.-%, based on the total weight of the reactant stream, wherein the concentration of aldehydes in the reactant stream is not lower than 10 ppm, preferably not lower than 25 ppm, in particular not lower than 50 ppm, or not lower than 100 ppm, based on the total weight of the reactant stream.
2. The process according to embodiment 1, wherein step a) comprises introducing, preferably by mixing and injecting or by spraying, the at least one formaldehyde source and isobutylene into a reactor, optionally through a plurality of nozzles operated in parallel, and reacting the formaldehyde source and isobutylene under supercritical conditions, wherein the reactor preferably comprises a vertically disposed vessel, a sidewall, an upper portion and a lower portion.
3. The process according to embodiment 2 wherein the formaldehyde source and isobutylene are introduced, for example injected or sprayed, into a mixing chamber of the reactor disposed in the upper portion and a fluid comprising formaldehyde and/or isobutylene and/or isoprenol is passed from the mixing chamber into a post-reaction chamber disposed in the lower portion.
4. The process according to embodiment 2 or 3, comprising providing draft tubes arranged essentially concentrically underneath each of the nozzles in the mixing chamber, the draft tubes providing downcomer conduits within the draft tubes and a riser conduit outside of the draft tubes, so that the formaldehyde source and isobutylene introduced, for example injected or sprayed, through the nozzles travel generally downward in the downcomer conduits, a fluid comprising formaldehyde and/or isobutylene and/or isoprenol is then diverted in a generally upward direction in the riser conduit, and the fluid is back-mixed with the introduced, for example injected or sprayed, formaldehyde source and isobutylene.
5. The process according to any one of embodiments 1 to 4, wherein step a) comprises introducing, preferably by mixing and injecting, or by spraying, the formaldehyde source and isobutylene into an internal loop reactor through at least one nozzle into first conduit(s), the internal loop reactor comprising:
   a vertically disposed cylindrical vessel comprising a sidewall;
   at least one draft tube having a tube inlet end and a tube outlet end, arranged vertically within the vessel, the draft tube(s) being arranged concentrically to the nozzle(s), and having an inner surface and an outer surface, wherein the draft tube(s) provide(s) the first conduit(s) within the draft tube(s), and a second conduit outside of the draft tube(s) and within the sidewall, the first conduit(s) being in fluid communication with the second conduit;
   reactor fluid outlet means;
wherein the inner surface of the draft tube(s) convexly curves so that the first conduit(s) exhibit(s) an annular constriction of the cross-section between the tube inlet end and the tube outlet end; wherein the constriction is located closer to the tube inlet end; wherein the convex curvature of the inner surface of the draft tube(s) extends over at least 70%, preferably at least 80%, most preferably at least 90% of the length of the draft tube; and
wherein the outer surface of the draft tube(s) convexly curves so that the draft tube(s) exhibit(s) a circumferential protuberance between the tube inlet end and the tube outlet end, which circumferential protuberance is preferably located closer to the tube outlet end; wherein the convex curvature of the outer surface of the draft tube(s) extends over at least 70%, preferably at least 80%, most preferably at least 90% of the length of the draft tube; and
wherein the edges of the draft tube(s) are rounded so that the formaldehyde source and isobutylene introduced, for example injected or sprayed, through the nozzles travel generally downward in the first conduit(s) to obtain a reacted fluid, the reacted fluid is then diverted in the opposite direction so as to travel through the second conduit and is subsequently back-mixed with the introduced, for example injected or sprayed, fluid.
6. The process according to any one of embodiments 1 to 5, wherein reacting the formaldehyde source and isobutylene comprises heat-exchanging a stream of hot isoprenol withdrawn from the reactor with an isobutylene stream directed to the reactor;
   wherein heat-exchanging is performed in one or more shell-and-tube heat exchangers; each of the heat exchangers comprising a plurality of tubes and a shell-side heat exchange passage;
   wherein the hot isoprenol is directed through the tubes of the heat exchangers; and the isobutylene is guided through the shell-side passage, and in case of more than one heat exchangers at least two of the heat exchangers are connected in series with regard to both the shell-side flow and the tube-side flow.
7. The process according to any one of embodiments 1 to 6, wherein isoprenol obtained in step a) is purified by subjecting a stream of crude isoprenol containing isoprenol, water and formaldehyde, or an isoprenol containing fraction thereof, to distillation in a low-boiler separation tower operated at a pressure of 2 bara or higher, preferably 2.5 bara or higher, to obtain a distillate stream containing aqueous formaldehyde and a bottoms stream containing isoprenol essentially free of formaldehyde.
8. The process according to any one of embodiments 1 to 7, wherein step c) comprises isomerizing isoprenol to prenol, and oxidative dehydrogenation of prenol to prenal, and wherein prior to oxidative dehydrogenation the prenol is treated to remove organically bound nitrogen from the prenol by contacting the prenol with a weakly acidic solid adsorbent.
9. The process according to any one of embodiments 1 to 8, wherein step c) comprises oxidative dehydrogenation of (iso)prenol by bringing a gaseous reactant stream comprising (iso)prenol into contact with a silver-containing heterogeneous catalyst in the presence of molecular oxygen.
10. The process according to embodiment 9, wherein step c-i) is characterized by maintaining in the reactant stream a weight ratio of aldehydes, preferably prenal and/or formaldehyde to isoprenol of less than 0.04,
   wherein the step b) is characterized by maintaining in the reactant stream a concentration of aldehydes of less than 0.5 wt.-%, preferably less than 0.4 wt.-%, in particular less than 0.3 wt.-%, or less than 0.25 wt.-%, based on the total weight of the reactant stream, wherein optionally the concentration of aldehydes in the reactant stream is not lower than 10 ppm, preferably not lower than 25 ppm, in particular not lower than 50 ppm, or not lower than 100 ppm, with respect to the total weight of the reactant stream.
11. The process according to any one of the preceding embodiments 1 to 10, wherein oxidative dehydrogenation of step c) is carried out by passing the (iso)prenol through a plurality of reaction tubes of a shell-and-tube heat exchange reactor comprising:
   a shell-side heat exchange passage for circulating a heat transfer medium and a reaction passage comprising the plurality of reaction tubes;
   an inlet for introducing the reactant stream to the reaction passage; and
   an outlet from the reaction passage for recovering an effluent stream from the reaction tubes;
   wherein the reaction tubes comprise
   a reactant pre-heating zone adjacent to the inlet, and
   a reaction zone downstream of the reactant pre-heating zone, the reaction zone having a catalytically active wire matrix insert having silver at least on a part of its surface.
12. The process of embodiment 11, wherein the catalytically active wire matrix inserts comprise an elongated core having a plurality of wire loops extending from the elongated core, wherein the wire loops are longitudinally arranged and helically shifted, and wherein the wire loops comprise a massive silver wire.
13. The process according to any one of embodiments 1 to 12, wherein step d) comprises continuously condensing prenol with prenal in the presence of at least one condensation catalyst in a reaction column while continuously withdrawing an acetal fraction comprising diprenyl acetal of prenal from the reaction column.
14. The process according to any one of embodiments 1 to 13, wherein step e) comprises continuously subjecting the acetal fraction in a cleaving column to cleaving conditions in the presence of at least one cleaving catalyst while continuously withdrawing from the cleaving column a cleaving fraction containing at least one of prenyl (3-methyl-butadienyl) ether and 2,4,4-trimethyl-3-formyl-1,5-hexadiene, and optionally containing citral; and reacting the cleaving fraction in a plug-flow type reactor to obtain citral.
15. The process according to embodiment 14, characterized in that the conversion rate of diprenyl acetal of prenal is maintained at above 90% and below 100%.
16. The process according to embodiment 14 or 15, wherein the cleaving catalyst is phosphoric acid, and wherein the concentration of the phosphoric acid in the bottoms of the cleaving column is above 100 ppm and below 1500 ppm.
17. The process according to any one of embodiments 14 to 16, wherein the condensation catalyst is nitric acid, and the concentration of the nitric acid is below 500 ppm.
18. The process according to any one of embodiments 14 to 17, comprising recycling prenol obtained in step e) to step d);
   wherein the concentration of 2,4,4-trimethyl-3-formyl-1,5-hexadiene of the prenol recycled from step e) into step d) is controlled such that the concentration of 2,4,4-trimethyl-3-formyl-1,5-hexadiene in step d) is below 1 wt.-%, relative to the total weight of prenol and prenal; and
   wherein the concentration of citral of the prenol recycled from step e) into step d) is controlled such that the concentration of citral in step d) is below 1 wt.-%, relative to the total weight of prenol and prenal.
19. The process according to any one of embodiments 1 to 18, wherein the aldehydes in the reactant stream consist of or comprise formaldehyde, and wherein the concentration of formaldehyde is less than 0.2 wt.-%, even more preferably less than 0.15 wt.-%, in particular less than 0.1 wt.-%, or less than 0.05 wt.-%, more particularly less than 0.025 wt.-%, even more particularly less than 0.02 wt.-%, based on the total weight of the reactant stream, optionally not less than 10 ppm, preferably not lower than 25 ppm, in particular not lower than 50 ppm, or not lower than 100 ppm, based on the total weight of the reactant stream.
20. The process according to any one of the embodiments 1 to 19, wherein the process includes separating an unreacted isoprenol stream from the prenol containing product stream, optionally removing at least some aldehydes, preferably some formaldehyde and/or prenal from the unreacted isoprenol stream, followed by combining the unreacted isoprenol stream with a fresh isoprenol stream to form the reactant stream, wherein the concentration of aldehydes, preferably formaldehyde and/or prenal, in the reactant stream is maintained at less than 0.5 wt.-%, preferably less than 0.4 wt.-%, in particular less than 0.3 wt.-%, or less than 0.25 wt.-%, based on the total weight of the reactant stream, wherein the concentration of aldehydes, preferably formaldehyde and/or prenal, in the reactant stream is not lower than 10 ppm, preferably not lower than ppm, in particular not lower than 50 ppm, or not lower than 100 ppm, with respect to the total weight of the reactant stream.
21. The process according to any one of the embodiments 1 to 19, wherein the process includes separating an unreacted isoprenol stream from the prenol containing product stream, combining the unreacted isoprenol stream with a crude isoprenol stream containing isoprenol, water and aldehydes, and removing water, preferably water and aldehydes, from the combined stream to form the reactant stream.
22. The process according to embodiment 21, wherein removing aldehydes, preferably water and aldehydes, in particular water, formaldehyde and/or prenal from the combined stream comprises:
   (i) directing the combined stream to a first low-boiler separation tower operated at a pressure of 1.5 bara or lower, to obtain a first bottoms stream containing isoprenol and aldehydes, and a first distillate stream containing water and low-boilers;
   (ii) directing the first bottoms stream to a second low-boiler separation tower operated at a pressure of 2 bara or higher, to obtain a second distillate stream containing aqueous aldehydes, and a second bottoms stream containing isoprenol; and
   (iii) directing the second bottoms stream to a finishing tower to obtain a bottoms stream containing high-boilers, and the reactant stream as a distillate stream.
23. The process according to any one of embodiments 1 to 22, wherein step a) is performed in the presence of less than 0.5 mol-%, preferably less than 0.1 mol-%, more preferably less than 0.01 mol-% and most preferably less than 0.001 mol-% of compounds capable of inducing further reactions of isoprenol, especially at least one Bronsted acid compound.
24. The process according to embodiment 23, wherein the Bronsted acid compound is selected from the group consisting of hydroxides, carbonates and bicarbonates of alkali metals and alkaline earth metals, ammonia or organic amines, particularly ethylamine, trimethylamine, hexamethylenetetramine (urotropin), aniline, pyridine, or piperidine, and salts of acids which are weaker than formic acid.
25. The process according to embodiments 1 to 22, wherein in the step a) the Bronsted base compound is present in amounts of 0.001 to 10 wt.-%, particularly 0.01 to 1 wt.-%, based on the total weight of reaction mixture.
26. The process according to embodiments 1 to 25, wherein in the step a) the starting mixture of the reactants, after the Bronsted base compound has been added, has a pH value of from 7 to 11, preferably from 7 to 10, particularly from 7.5 to 9, as determined at 20° C. and 1 bar absolute.
27. The process according to embodiments 1 to 26, wherein in the step a) the formaldehyde source is an aqueous formaldehyde solution and the Bronsted base compound is preferably fed into the reactor dissolved in the aqueous formaldehyde solution.
28. The process according to embodiments 1 to 26, wherein in the step a) the formaldehyde source is an aqueous formaldehyde solution and the Bronsted base compound is separately fed into the reactor as an aqueous solution.

The invention is moreover illustrated by the attached drawings and the examples that follow.

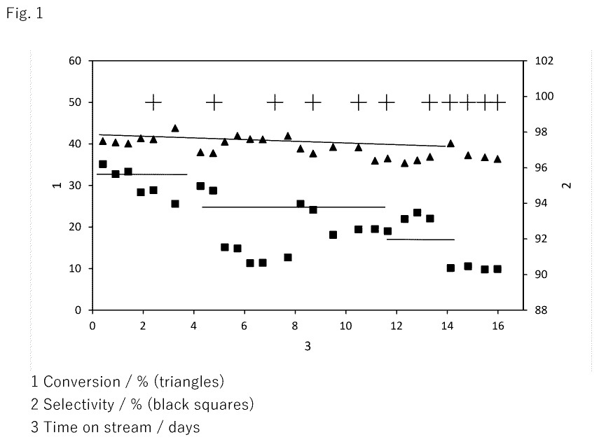

FIG. 1 shows conversion and selectivity over time on stream of an isoprenol oxidative dehydrogenation reaction.

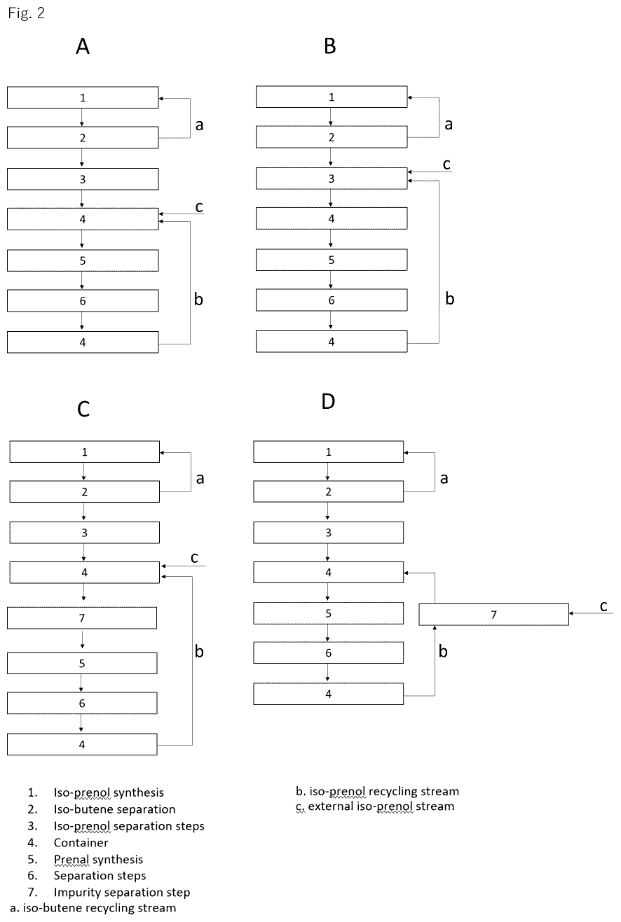

FIG. 2 schematically depicts Schemes A to D with different options to accommodate the recycling stream of unreacted isoprenol.

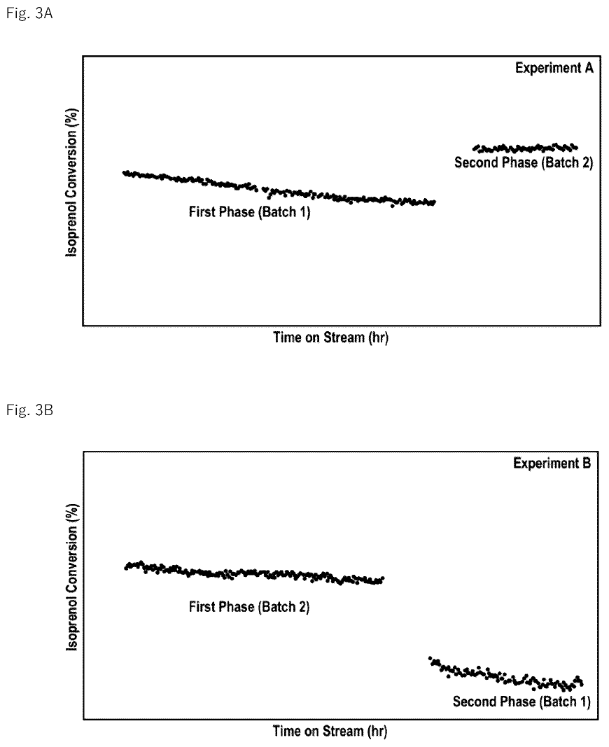

FIG. 3A indicates the relative isoprenol conversion during the first and second phase of Experiment A as described below, based on online gas chromatography measurements.

FIG. 3B indicates the relative isoprenol conversion during the first and second phase of Experiment B as described below, based on online gas chromatography measurements.

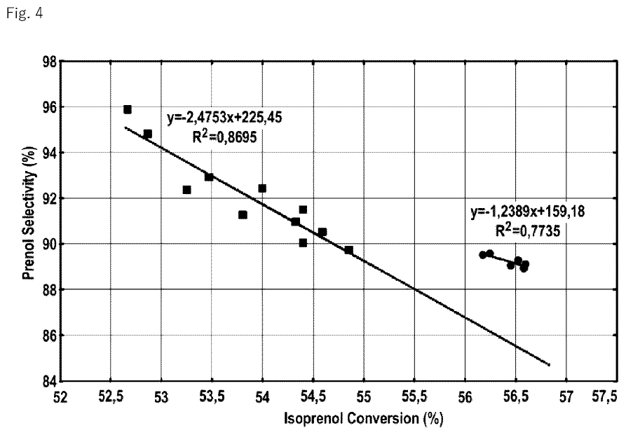

FIG. 4 shows the prenol selectivity relative to the isoprenol conversion for Experiment A as described below, including the extrapolation of the observed selectivity for Phase 1 (data points between 52.5% and 55% conversion) and Phase 2 (data points between 56% and 57% conversion) as indicated by the dotted line.

In accordance with this invention, maintaining in the reactant stream a certain weight ratio of formaldehyde to isoprenol can be accomplished in a number of ways as illustrated in FIG. 2. Scheme A shows how the recycling stream b is typically incorporated in a prior art process. Scheme B shows how the recycling stream b can be resent through the isoprenol separation steps of an existing isoprenol manufacturing unit. Scheme C shows the total reactor feed stream going through an additional impurity separation step, for example a distillation step. Scheme D shows only the recycling stream going through an additional impurity separation step, for example a distillation step.

### Example 1

Isoprenol oxidation is investigated in a mini plant reactor. The catalyst bed consists of a 30 cm packing of silver coated (5 wt.-%) steatite spheres (diameter 2 mm) in a stainless-steel tube (inner diameter 12 mm). The cooling jacket temperature is kept at 380° C. An isoprenol load of 300 g/h was adopted with 30 g/h of water and 92 L/h of air. The reactant stream is quenched through a water cooler. The condensate is analyzed offline by gas chromatography. The uncondensed gas stream is analyzed online by gas chromatography. Conversion and (iso)prenal selectivity are calculated using both analyses.

An experiment with isoprenol having formaldehyde concentrations increasing from 0, 2, 3 to 4 wt.-% was carried out.

FIG. 1 shows conversion and selectivity over time on stream. At day 5, formaldehyde concentration was increased to 2 wt.-%, on day 14 to 3 wt.-%. Regeneration cycles are marked with a plus sign at 50% conversion. With increasing formaldehyde concentration, drops in selectivity are observed. At initial time on stream, a selectivity drop of about 1.5 percentage points is observed per wt.-% of formaldehyde contained in the feed. The regeneration cycles were more often required with increasing formaldehyde concentration. A still higher frequency of regeneration cycles was necessary at 4 wt.-% of formaldehyde.

### Example 2

Isoprenol having varying concentrations of formaldehyde as in example 1 is subjected to isomerization. The isomerization reaction is conducted in accordance with WO 2008/037693. It was found that also the isomerization proceeds advantageously with isoprenol having low/reduced formaldehyde content.

In accordance with this invention, maintaining in the reactant stream a certain concentration of aldehydes, in particular of formaldehyde and/or prenal, especially of formaldehyde, can be accomplished in a number of ways as illustrated in FIG. 2. Scheme A shows how the recycling stream b is typically incorporated in a prior art process. Scheme B shows how the recycling stream b can be resent through the isoprenol separation steps of an existing isoprenol manufacturing unit. Scheme C shows the total reactor feed stream going through an additional impurity separation step, for example a distillation step. Scheme D shows only the recycling stream going through an additional impurity separation step, for example a distillation step.

### Experiments A and B

Isoprenol batches having varying concentrations of aldehydes, i.e., prenal and formaldehyde were subjected to isomerization. The compositions of the batches of isoprenol, as determined via gas chromatography (GC) analysis, are shown below, except for the amount of prenal and formaldehyde of batch 1, which was provided via quality control from the isoprenol production.

| | Aldehydes | Prenal | Formaldehyde |
|---|---|---|---|
| Batch | [wt.-%] | [wt.-%] | [wt.-%] |
| 1 | 0.57 to 0.87 | 0.37 | 0.2 to 0.5 |
| 2 | 0.17 | 0.09 | 0.08 |

Isomerization of the isoprenol batches was conducted as follows: A double-walled glass reactor was filled with 100 mL (48.8 g) of an isomerization catalyst (fixed bed catalyst containing Pd/Se supported on SiO₂). The top of the reactor was connected to a phase separator, the bottom of which phase separator was connected to the reactor bottom via an external circulation pump. The reactor temperature was regulated via an external oil jacket. Before each experiment, the catalyst was dried under a nitrogen flow at 120° C. for 16 h, reduced using a 1:1 (vol:vol) mixture of hydrogen and nitrogen at 120° C. for 2 h, and then flushed with nitrogen at 70° C. for 16 h.

During start-up, a feed of the respective isoprenol batch was pumped at 100 g/h from the bottom to the top of the reactor, in the same direction of flow as co-fed hydrogen (6 NI/h, 1.2 bara), released through a bubble frit at the bottom of the reactor. A product stream from the top of the reactor was fed to the phase separator.

After a 24 h start-up phase, sufficient product had accumulated in the phase separator to begin recycling through the circulation pump. The feed supply was reduced to 70 g/h and the liquid phase of the product stream obtained in the phase separator was recycled to the bottoms of the reactor via at a rate of 150 g/h. At the same time, the reactor pressure was increased to 1.5 bara and the temperature raised to 80° C. These conditions were maintained for the remainder of the experiment.

In the course of the experiments, liquid samples were taken from the product stream taken from the top of the reactor and analyzed by offline GC. Moreover, online GC measurements were continually performed.

Two feed-switch experiments were conducted, as indicated below, whereby the reactor was supplied with one batch for about 6 to 10 days (first phase) before switching to the other batch and measuring until a new steady-state was reached (about 2 to 5 days, second phase). In this way, it was possible to directly compare the performance of the two batches.

| Experiment | Start-Up Phase | First Phase | Second Phase |
|---|---|---|---|
| A | Batch 1 | Batch 1 | Batch 2 |
| B | Batch 2 | Batch 2 | Batch 1 |

Conversion of isoprenol (%) was calculated as 100×([wt.-% isoprenol in feed]−[wt.-% isoprenol in product])/[wt.-% isoprenol in feed]. Selectivity for prenol (%) was calculated as 100×([wt.-% prenol in product]−[wt.-% prenol in feed])/([wt.-% isoprenol in feed]−[wt.-% isoprenol in product]). The prenol yield was calculated as (prenol selectivity×isoprenol conversion)/100.

The mean averages of isoprenol conversion as determined via offline GC are shown in the table below.

| Experiment | Start-Up Phase | First Phase | Second Phase |
|---|---|---|---|
| A | Batch 1 | Batch 1 | Batch 2 |
| | Conversion: 36.9% | Conversion: 53.8% | Conversion: 56.5% |
| B | Batch 2 | Batch 2 | Batch 1 |
| | Conversion: 42.4% | Conversion: 59.0% | Conversion: 53.6% |

It was found that when high-purity isoprenol of Batch 2 was used as the feed, the catalyst was more active and the conversion of isoprenol was higher than when feeding Batch 1. Moreover, the prenol yield higher. The higher conversion is also evident from the results of the online GC measurements, as indicated in FIGS. 3A and 3B. Said figures also show that the rate of deactivation was greater when feeding Batch 1, suggesting that the catalyst lost its activity more rapidly in the presence of this batch of isoprenol.

Further, it was found that the prenol selectivity when using high-purity isoprenol of Batch 2, when extrapolated relative to the isoprenol conversion, is higher than for the isoprenol of Batch 1, as depicted in FIG. 4.

FIG. 4 shows the Prenol selectivity as a function of isoprenol conversion, as determined by offline GC data for Experiment A. Square dots represent the isoprenol batch 1 (normal quality isoprenol); circle dots the isoprenol batch 2 (the high-purity isoprenol). The offline GC analyses performed when feeding the isoprenol batch 1 (red points in FIG. 4) can be well fitted to a linear trendline. This trendline shows that, under these conditions, as isoprenol conversion decreased with extended function of time on stream (TOS), the prenol selectivity increased at a rate of 2.5% of selectivity per 1% of conversion. Extrapolating this trendline to the higher conversions achieved with the high-purity feed reveals a predicted selectivity towards prenol of ~86%. However, prenol selectivity of >89% were recorded with the isoprenol batch 2, which is the high-purity feed (circle dots in FIG. 4), showing that a more selective catalysis was achieved after switching to the higher purity feed.

Interestingly, using batch 2 with lower aldehyde values compared to batch 1 during the start-up phase resulted in a higher activity level of the catalyst right from the start, and hence the conversion rate after approximately 24 h and before the first phase and during the first phase was higher when batch 2 was used from the start, compared to the experiment when batch 1 was used from the start through the start-up and first phase.

1.-37. (canceled)

38. Process for the preparation prenal comprising a step of subjecting isoprenol to oxidative dehydrogenation so as to obtain prenal and/or isoprenal by bringing a reactant stream comprising isoprenol into contact with at least one heterogeneous oxidative dehydrogenation catalyst, in the presence of molecular oxygen, and optionally isomerizing at least part of the isoprenal to prenal,
wherein the step is characterized by maintaining in the reactant stream a weight ratio of aldehydes to isoprenol of less than 0.04.

39. Process for the preparation of diprenyl acetal of prenal comprising the step of condensing prenal obtained according to claim 38 with prenol.

40. The process according to claim 39, wherein the prenol is obtained from isomerizing isoprenol by bringing a reactant stream comprising isoprenol into contact with at least one heterogeneous isomerization catalyst, preferably in the presence of hydrogen.

41. The process according to claim 40, wherein the step of isomerizing isoprenol is characterized by maintaining in the reactant stream a concentration of aldehydes of less than 0.5 wt.-% based on the total weight of the reactant stream, and, optionally, the concentration of aldehydes in the reactant stream is not lower than 10 ppm based on the total weight of the reactant stream.

42. The process according to claim 39, comprising the steps of:
a) reacting at least one formaldehyde source and isobutylene to obtain isoprenol;
b) isomerizing isoprenol obtained in step a) to obtain prenol by bringing a reactant stream comprising isoprenol into contact with at least one heterogeneous isomerization catalyst, preferably in the presence of hydrogen;
c) providing the prenal; and
d) condensing prenol obtained in step b) with prenal obtained in step c) to obtain diprenyl acetal of prenal.

43. Process for the preparation of 3,7-dimethyl-octa-2,6-dienal (citral) comprising a step subjecting diprenyl acetal of prenal obtained according to claim 39 to cleaving conditions to obtain citral via prenyl (3-methyl-butadienyl) ether and 2,4,4-trimethyl-3-formyl-1,5-hexadiene.

44. Process for the preparation of 3,7-dimethyl-octa-2,6-dienal (citral) comprising the steps of:
a) reacting at least one formaldehyde source and isobutylene to obtain isoprenol;
b) isomerizing isoprenol obtained in step a) to obtain prenol by bringing a reactant stream comprising isoprenol into contact with at least one heterogeneous isomerization catalyst;
c) providing prenal by at least one of c-i) and c-ii):
c-i) subjecting isoprenol obtained in step a) to oxidative dehydrogenation so as to obtain prenal and/or isoprenal by bringing a reactant stream comprising isoprenol into contact with at least one heterogeneous oxidative dehydrogenation catalyst, in the presence of molecular oxygen, and optionally isomerizing at least part of the isoprenal to prenal;
c-ii) oxidizing prenol obtained in step b) so as to obtain prenal by bringing a reactant stream comprising prenol into contact with at least one oxidant and at least one oxidation catalyst;
d) condensing prenol obtained in step b) with prenal obtained in step c) to obtain diprenyl acetal of prenal; and
e) subjecting diprenyl acetal of prenal obtained in step d) to cleaving conditions to obtain citral via prenyl (3-methyl-butadienyl) ether and 2,4,4-trimethyl-3-formyl-1,5-hexadiene;
wherein step c-i) is characterized by maintaining in the reactant stream a weight ratio of aldehydes to isoprenol of less than 0.04, and
optionally, step b) is characterized by maintaining in the reactant stream a concentration of aldehydes of less than 0.5 wt.-%, based on the total weight of the reactant stream, and,
optionally, the concentration of aldehydes in the reactant stream is not lower than 10 ppm, based on the total weight of the reactant stream.

45. The process according to claim 38, wherein isoprenol is purified by subjecting a stream of crude isoprenol containing isoprenol, water and formaldehyde, or an isoprenol containing fraction thereof, to distillation in a low-boiler separation tower operated at a pressure of 2 bara or higher to obtain a distillate stream containing aqueous formaldehyde and a bottoms stream containing isoprenol essentially free of formaldehyde.

46. The process according to claim 38, wherein the reactant stream comprising isoprenol is gaseous and at least one heterogeneous oxidative dehydrogenation catalyst is a silver-containing heterogeneous oxidative dehydrogenation catalyst.

47. The process according to claim 38, wherein oxidative dehydrogenation is carried out by passing the isoprenol through a plurality of reaction tubes of a shell-and-tube heat exchange reactor comprising
a shell-side heat exchange passage for circulating a heat transfer medium and a reaction passage comprising the plurality of reaction tubes;
an inlet for introducing the reactant stream to the reaction passage; and
an outlet from the reaction passage for recovering an effluent stream from the reaction tubes;
wherein the reaction tubes comprise:
a reactant pre-heating zone adjacent to the inlet, and
a reaction zone downstream of the reactant pre-heating zone, the reaction zone having a catalytically active wire matrix insert having silver at least on a part of its surface.

48. The process according to claim 47, wherein the catalytically active wire matrix inserts comprise an elongated core having a plurality of wire loops extending from the elongated core, wherein the wire loops are longitudinally arranged and helically shifted, and wherein the wire loops comprise a massive silver wire.

49. The process according to claim 43, wherein obtaining citral comprises continuously subjecting the acetal fraction in a cleaving column to cleaving conditions in the presence of at least one cleaving catalyst while continuously withdrawing from the cleaving column a cleaving fraction containing at least one of prenyl (3-methyl-butadienyl) ether and 2,4,4-trimethyl-3-formyl-1,5-hexadiene, and optionally containing citral; and reacting the cleaving fraction in a plug-flow type reactor to obtain citral.

50. Process for the preparation of prenol by bringing a reactant stream comprising isoprenol into contact with at least one heterogeneous isomerization catalyst to obtain a prenol containing product stream, characterized by maintaining in the reactant stream a concentration of aldehydes of less than 0.5 wt.-% based on the total weight of the reactant stream, wherein the concentration of aldehydes in the reactant stream is not lower than 10 ppm with respect to the total weight of the reactant stream.

51. The process according to claim 50, wherein the isoprenol is obtained from reacting at least one formaldehyde source and isobutylene.

52. The process according to claim 50, wherein the process includes separating an unreacted isoprenol stream from the prenol containing product stream, optionally removing at least some aldehydes, from the unreacted isoprenol stream, followed by combining the unreacted isoprenol stream with a fresh isoprenol stream to form the reactant stream.

53. The process according to claim 50, wherein the process includes separating an unreacted isoprenol stream from the prenol containing product stream, combining the unreacted isoprenol stream with a crude isoprenol stream containing isoprenol, water and aldehydes, and removing water from the combined stream to form the reactant stream.

54. Process for the preparation of diprenyl acetal of prenal comprising the step of condensing prenol obtained according to claim 50 with prenal.

55. The process according to claim 54, comprising the steps of:
a) reacting at least one formaldehyde source and isobutylene to obtain isoprenol;
b) isomerizing isoprenol obtained in step a) to obtain prenol by bringing a reactant stream comprising isoprenol into contact with at least one heterogeneous isomerization catalyst;
c) providing prenal by at least one of c-i) and c-ii):
c-i) subjecting isoprenol obtained in step a) to oxidative dehydrogenation so as to obtain prenal and/or isoprenal by bringing a reactant stream comprising isoprenol into contact with at least one heterogeneous oxidative dehydrogenation catalyst, in the presence of molecular oxygen, and optionally isomerizing at least part of the isoprenal to prenal;
c-ii) oxidizing prenol obtained in step b) so as to obtain prenal by bringing a reactant stream comprising prenol into contact with at least one oxidant and at least one oxidation catalyst; and
d) condensing prenol obtained in step b) with prenal obtained in step c) to obtain diprenyl acetal of prenal.

56. Process for the preparation of 3,7-dimethyl-octa-2,6-dienal (citral) comprising a step subjecting diprenyl acetal of prenal obtained according to claim 55 to cleaving conditions to obtain citral via prenyl (3-methyl-butadienyl) ether and 2,4,4-trimethyl-3-formyl-1,5-hexadiene.

57. Process for the preparation of 3,7-dimethyl-octa-2,6-dienal (citral) comprising the steps of:
a) reacting at least one formaldehyde source and isobutylene to obtain isoprenol;
b) isomerizing isoprenol obtained in step a) to obtain prenol by bringing a reactant stream comprising isoprenol into contact with at least one heterogeneous isomerization catalyst;
c) providing prenal by at least one of c-i) and c-ii):
c-i) subjecting isoprenol obtained in step a) to oxidative dehydrogenation so as to obtain prenal and/or isoprenal by bringing a reactant stream comprising isoprenol into contact with at least one heterogeneous oxidative dehydrogenation catalyst, in the presence of molecular oxygen, and optionally isomerizing at least part of the isoprenal to prenal;
c-ii) oxidizing prenol obtained in step b) so as to obtain prenal by bringing a reactant stream comprising prenol into contact with at least one oxidant and at least one oxidation catalyst;
d) condensing prenol obtained in step b) with prenal obtained in step c) to obtain diprenyl acetal of prenal; and
e) subjecting diprenyl acetal of prenal obtained in step d) to cleaving conditions to obtain citral via prenyl (3-methyl-butadienyl) ether and 2,4,4-trimethyl-3-formyl-1,5-hexadiene;
wherein step b) is characterized by maintaining in the reactant stream a concentration of aldehydes of less than 0.5 wt.-% based on the total weight of the reactant stream, and,
optionally, the concentration of aldehydes in the reactant stream is not lower than 10 ppm based on the total weight of the reactant stream; and
optionally wherein step c-i) is characterized by maintaining in the reactant stream a weight ratio of aldehydes to isoprenol of less than 0.04.
